(19) United States
(12) Patent Application Publication
(10) Pub. No.: US 2023/0064034 A1
(43) Pub. Date: Mar. 2, 2023

(54) LIGHT STABLE LIQUID DISINFECTANT COMPOSITIONS

(71) Applicant: Onyx Lotus, LLC, Dover, DE (US)

(72) Inventor: Amyn Nanjee, Dover, DE (US)

(21) Appl. No.: 17/888,469

(22) Filed: Aug. 15, 2022

Related U.S. Application Data
(60) Provisional application No. 63/233,161, filed on Aug. 13, 2021.
(60) Provisional application No. 63/234,593, filed on Aug. 18, 2021.

(51) Int. Cl. A01N 59/20; A01N 25/04; A01N 25/30

(52) U.S. Cl. CPC A01N 59/20; A01N 25/04; A01N 25/30

(57) ABSTRACT

The present invention provides a liquid disinfectant composition, method for preparing a liquid disinfectant composition, and methods of disinfecting of a surface or article using the liquid disinfectant composition. These liquid metal ion compositions are light stable, non-toxic, and non-corrosive, achieves a greater than 99% kill rate against a variety of pathogens for up to 60 days on a variety of surfaces, and do not contain nanoparticles.

### CROSS-REFERENCE TO RELATED APPLICATIONS

This application claims priority to U.S. Provisional Application 63/233,161 filed on Aug. 13, 2021 and U.S. Provisional Application 63/234,593 filed on Aug. 18, 2021, both of which are hereby incorporated by reference herein.

### FIELD OF THE INVENTION

The present disclosure generally relates to liquid disinfectant compositions, methods of preparing these liquid disinfectant compositions, and methods of using liquid disinfectant compositions. In particular, the present disclosure relates to liquid disinfectant compositions comprising at least one metal ion and at least one hydrophilic polymer in a solvent, which may include at least one chelating agent, at least one surfactant, or a combination of at least one chelating agent and at least one surfactant, and at least one additive. These liquid disinfectant compositions are light stable, heat stable, non-toxic, and non-corrosive, achieve a greater than 99% kill rate on a variety of pathogens, and maintain pathogen sterility for up to 60 days on a variety of surfaces and articles. Additionally, the liquid disinfectant compositions do not contain nanoparticles.

### BACKGROUND OF THE INVENTION

Effective control of pathogens, such as viruses, bacteria, fungus, and mold, using various commercial disinfectants, such as bleach, sprays (e.g., Lysol®), and aqueous compositions (e.g., Pine Sol®) has been performed for a number of years. Through the use of commercial disinfectants, whether sprays or wipes, an adequate number of bacteria, fungus, and mold can be effectively reduced. Yet, despite this immediate reduction, the bacteria, fungus, or mold can quickly repopulate once the disinfectant has dissipated or evaporated from the surface. Further, these commercial disinfectants generally have little or no effect on viruses and in some cases are considered corrosive and toxic.

Therefore, there is a desire to prevent the transmission of pathogens. One method of reducing pathogen transmission is to reduce the period of human vulnerability to infection by reducing the period of viability of pathogens on solids and surfaces.

Surfaces may be treated with chemical biocides, such as bleach, quaternary ammoniums salts, or UV light, to disinfect bacteria and destroy viruses within a matter of minutes. Problematically, some of these biocides can be considered toxic and corrosive.

Alternatively, antimicrobial coatings may be applied to a surface to kill bacteria and/or destroy viruses, however, these coatings are limited by a low concentration of biocides at the surface due to slow biocide transport. The slow diffusion of biocides through the solid coating results in limited availability and can generally require up to two hours to kill 99.9 wt % of bacteria and/or deactivate 99.9 wt % of viruses. Light (e.g., UV or natural light) can also degrade these biocides and reduce the effectiveness of these antimicrobial effectiveness. Unfortunately, these biocides do not maintain the kill rate for an extended period of time due to the reasons disclosed above.

Therefore, there is a need for a light stable, heat stable, non-toxic, and non-corrosive liquid disinfectant composition that is not only effective against different pathogens (e.g., bacteria, mold, fungi, and viruses) but also provides increased efficacy (greater than 99% pathogen kill rate) on a variety of surfaces over an extended period of time.

### SUMMARY OF THE INVENTION

One aspect of the present disclosure encompasses a liquid disinfectant composition for killing pathogens and/or maintaining the pathogenic sterility on a surface or an article comprising: (a) 0.001 weight % (wt %) to 5.0 wt % of at least one metal ion; (b) 0.1 wt % to 5.0 wt % of at least one hydrophilic polymer; and (c) 90 wt % to 99.9 wt % of at least one solvent; wherein the liquid disinfectant does not comprise nanoparticles. The liquid disinfectant composition utilizes the hydrophilic polymer to prevent oxidation and/or moisture contact of the at least one metal ion and maintains contact with a variety of surfaces and/or articles. The composition achieves a kill rate of greater than 99% on a variety of pathogens in a period of time of 5 minutes or less and is light stable, non-corrosive, and non-toxic.

Another aspect of the present disclosure encompasses a method for preparing a liquid disinfectant composition. The method comprises: (a) contacting at least one metal ion with at least one solvent to form a mixture; and (b) contacting the mixture from step (a) with at least one hydrophilic polymer to form the liquid disinfectant composition; wherein the liquid disinfectant composition does not comprise nanoparticles.

Yet another aspect of the present disclosure encompasses a method of cleaning and/or disinfecting the surface or an article. The method comprises contacting the surface of the article with a liquid disinfectant composition, the liquid disinfectant composition comprising: (a) at least one metal ion and (b) at least one hydrophilic polymer in a least one solvent; wherein the liquid disinfectant does not comprise nanoparticles. The liquid disinfectant composition utilizes the hydrophilic polymer to prevent oxidation and/or moisture contact of the at least one metal ion and maintains contact with a variety of surfaces and/or articles. The composition achieves a kill rate greater than 99% on a variety of pathogens in a period of time of 5 minutes or less and is light stable, non-corrosive, and non-toxic.

Other features and iterations of the invention are described in more detail below.

### DETAILED DESCRIPTION OF THE INVENTION

The present disclosure is based in part on the surprising discovery that the liquid disinfectant compositions kill a variety of pathogens. The kill rate is greater than 99% and pathogen sterility is maintained for up to 60 days. Importantly, these compositions are light stable, heat stable, economical, easily prepared, non-toxic to humans, non-corrosive to humans, exhibit antimicrobial properties, antibacterial properties, antiviral properties, antifungal properties, or a combination thereof. The compositions do not contain nanoparticles.

### (I) Liquid Disinfectant Compositions

The present invention relates to a liquid disinfectant composition that kills pathogens, viruses, and bacteria. The composition includes a metal salt and a hydrophilic polymer whereby the metal salt destroys the pathogen by disrupting or enveloping the pathogen cell wall. The hydrophilic polymer is used to attach the metal ion to the surface of the pathogen so that it remains in contact with the pathogen for a period of time. The composition further includes a chelating agent which is used to prevent degradation of the metal salt. The composition additionally includes a surfactant which provides greater solubility of low solubility water salts.

One aspect of the present disclosure encompasses liquid disinfectant compositions. The liquid disinfectant compositions comprise: (a) at least one metal salt and (b) a hydrophilic polymer in at least one solvent. The liquid disinfectant composition may include at least one chelating agent, at least one surfactant, or a combination of at least one chelating agent and at least one surfactant, and at least one additive. Generally, these liquid disinfectant compositions are easily prepared, do not contain nanoparticles, are light stable, heat stable, non-toxic, non-corrosive, and exhibit antimicrobial properties, antibacterial properties, antifungal properties, antiviral properties, or a combination thereof by killing more than 99% of pathogens as well as maintaining their effectiveness on a surface of an article for up to 60 days. In some embodiments, pathogenic sterility is maintained on a surface for up to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

### (a) Metal Salt

The liquid disinfectant composition includes at least one metal salt. The metal salt is preferably a water-soluble metal salt which releases a metal ion in at least one solvent. The metal salt may be a transition metal salt that imparts disinfectant properties.

Silver, as well as other metal ions, such as copper, zinc, gold, cobalt, nickel, zirconium, molybdenum, and palladium ions possess antimicrobial properties, antibacterial properties, and antifungal properties. Salts of these ions are considered to be active antimicrobial agents, antibacterial agents, and antifungal agents as long as a portion of the metal ion dissociates from the metal salt in a solvent (such as water, brine, or a polar protic solvent).

The metal ions react with pathogens at low ppm (parts per million) concentrations in various ways, such as binding to the wall of the pathogen to block substances from coming in or out of the pathogen, releasing active oxygen species which interact with the DNA or RNA of the pathogen to inhibit replication of the pathogen, and by transporting within the cell of the pathogen to block the respiratory system of the pathogen to destroy energy production. By contacting the pathogen with the liquid disinfectant composition, the metal ion releases reactive oxygen species. Non-limiting examples of reactive oxygen species may be oxygen, a superoxide anion, a peroxide anion, a hydroxyl radical, or combinations thereof. These reactive oxygen species, once in contact with a pathogen, can cause damage to cells through oxidative damage. The metal ions present a positively charged surface, which interact with the negatively charged pathogen membrane and cause physical damage. This membrane permeability can cause a disruption by electrostatic interactions with the pathogen.

The metal salt is a transition metal salt that imparts disinfectant properties. Non-limiting examples of the transition metal salts which impart disinfectant properties may be selected from a group consisting of a silver salt, a copper salt, a zinc salt, a gold salt, a cobalt salt, a nickel salt, a zirconium salt, a molybdenum salt, a palladium salt, and combinations thereof. The anion of the metal salt may have an organic or an inorganic anion. Non-limiting examples of the metal salt may be silver nitrate, silver acetate, silver bromide, silver sulfate, silver citrate, silver oxalate, copper (II) acetate, copper (II) sulfate, copper (I) chloride (II) carbonate, zinc chloride, zinc nitrate, zinc acetate, zinc sulfate, gold acetate, gold chloride, cobalt (II) sulfate, cobalt (II) chloride, cobalt (II) nitrate, cobalt (II) carbonate, nickel chloride, nickel sulfate, zirconium (IV) nitrate, zirconium (IV) acetate, molybdenum (II) chloride, molybdenum (V) chloride, and palladium (II) chloride. In some embodiments, the liquid disinfectant composition includes a silver salt, a copper salt, a zinc salt, or a combination thereof. In one embodiment, the liquid disinfectant composition includes a silver salt. In another embodiment, the liquid disinfectant composition includes a copper salt. In still another embodiment, the liquid disinfectant composition includes a zinc salt. In yet another embodiment, the liquid disinfectant composition includes a silver salt and a zinc salt. In still another embodiment, the liquid disinfectant composition includes a copper salt and a zinc salt. In yet another embodiment, the liquid disinfectant composition includes a silver salt and a copper salt. In still another embodiment, the liquid disinfectant composition includes a silver salt, a copper salt, a zinc salt, or a combination thereof.

In these embodiments, the liquid disinfectant composition includes a silver salt which is capable of releasing a silver cation, such as Ag⁺ but potentially Ag²⁺, Ag³⁺ in addition to Ag⁺. Suitable, non-limiting examples of silver salts may be silver chloride, silver bromide, silver fluoride (AgF, AgF₂, and/or Ag₂F), silver iodide, silver citrate, silver lactate, silver phosphate, silver carbonate, silver sulfate, silver trifluoroacetate, silver perchlorate, silver acetate, silver nitrate, silver sulfide, silver oxide, silver perchlorate, silver sulfadiazine, and combinations thereof. In one embodiment, the silver salt is silver nitrate. In another embodiment, the silver salt is silver chloride.

In general, the metal salt may be present in an amount ranging from about 0.001 wt % to about 5.0 wt % based on the total weight of the liquid disinfectant composition. In various embodiments, the at least one metal salt may be present in an amount ranging from about 0.001 wt % to about 5.0 wt %, from about 0.01 wt % to about 4.0 wt %, from about 0.05 wt % to about 3.0 wt %, from about 0.1 wt % to about 2.5 wt %, or from about 1.0 wt % to about 2.0 wt % based on the total weight of the liquid disinfectant composition.

In general, the metal salt may be present in an amount ranging from about 0.001 mole % to about 0.05 mole %. In various embodiments, the metal salt may be present in an amount ranging from about 0.001 mole % to about 0.05 mole %, from about 0.003 mole % to about 0.03 mole %, or from about 0.007 mole % to about 0.01.

### (b) Hydrophilic Polymer

The disinfectant composition includes at least one hydrophilic polymer. The hydrophilic polymer interacts with the metal salt or the chelated metal ion and provides stability to the metal salt and the hydrophilic polymer to prevent oxygen and/or moisture from interacting with the metal ion. The hydrophilic polymer has high polarity and propensity to form hydrogen bonds with various hydrogen donors such as phenols, carboxylic acids, anionic dyes, and inorganic salts. With this hydrogen bonding, the hydrophilic polymer interacts with the complex of the metal ion and the metal ions chelated complex through ionic and/or Van der Walls interactions of the oxygen atom on the hydrophilic polymer and prevents oxygen (O₂) and/or water from forming a metal oxide from interacting with the metal ion. This interaction not only stabilizes the complex but also increases the shelf-life of the liquid disinfectant composition.

Various hydrophilic polymers are widely known to impart antimicrobial properties to the composition, such as poly(vinyl pyrrolidone). By including the hydrophilic polymer in the disinfectant composition, the hydrophilic polymer provides a synergistic effect to the liquid disinfectant composition.

The hydrophilic polymer, as utilized in the liquid disinfectant composition, has a low evaporation rate. This property of the hydrophilic polymer allows the liquid disinfectant composition to remain connected to the article and the liquid disinfectant composition retains its potency over a 30-day period.

A wide range of hydrophilic polymers may be used in the liquid disinfectant composition. Suitable, non-limiting examples of hydrophilic polymers may be selected from a group consisting of a polyacrylamide, a poly(acrylamide-co-acrylic acid), poly(vinyl alcohol), poly(vinyl pyrrolidone) such as low and high molecular weight poly(vinyl pyrrolidone), poly(ethylene oxide), water soluble polyurethane, carboxy methyl cellulose, lipids such as glycerolipids, fatty acid lipid polymers, oligosaccharides, glycerols, or combinations thereof. In a certain embodiment, the hydrophilic polymer used in the liquid disinfectant composition is poly(vinylpyrrolidone) (PVP). In one embodiment, the hydrophilic polymer is poly(vinylpyrrolidone) K-30 or poly(vinylpyrrolidone) K-90.

Generally, the hydrophilic polymer can be present in an amount ranging from about 0.1 wt % to about 5.0 wt % based on the total weight of the liquid disinfectant composition. In various embodiments, the hydrophilic polymer can be present in an amount ranging from about 0.1 wt % to about 5.0 wt %, from about 0.1 wt % to about 4.0 wt %, from about 0.5 wt % to about 3.0 wt %, from about 0.5 wt % to about 2.5 wt %, or from about 1.0 wt % to about 2.0 wt % based on the total weight of the liquid disinfectant composition.

In general, the weight ratio of the hydrophilic polymer to the metal salt ranges from about 30.0:1.0 to about 70.0:1.0. In various embodiments, the weight ratio of the hydrophilic polymer to the metal salt ranges from about 30.0:1.0 to about 70.0:1.0, from about 35.0:1.0 to about 65.0:1.0, or from about 40.0:1.0 to about 60.0:1.0. In one embodiment, the weight ratio of the hydrophilic polymer to the metal salt is about 50.0:1.0.

Generally, the hydrophilic polymer may be present in an amount ranging from about 0.0000001 mole % to about 0.001 mole %. In various embodiments, the hydrophilic polymer may be present in an amount ranging from about 0.0000001 mole % to about 0.001 mole %, from about 0.0000003 mole % to about 0.0009 mole %, or from about 0.000001 mole % to about 0.0005 mole %.

### (c) Solvent

The liquid disinfectant composition comprises at least one solvent. The solvent can and will vary depending on the components used in the composition. In various embodiments, the solvent may be a polar protic solvent, a polar aprotic solvent, or combinations thereof. Suitable examples of polar protic solvents include, but are not limited to, water; alcohols such as methanol, ethanol, isopropanol, n-propanol, iso-butanol, n-butanol, s-butanol, t-butanol, and the like; diols such as ethylene glycol, propylene glycol; polyols such as glycerol, mannitol, sorbitol; and combinations thereof. Non-limiting examples of suitable polar aprotic solvents include but are not limited to acetonitrile N,N-dimethylacetamide (DMAC), N,N-dimethylformamide (DMF), dimethyl sulfoxide (DMSO), 1,4-dioxane, N-methyl-2-pyrrolidinone (NMP), hexamethylphosphoramide, N-methylacetamide, tetrahydrofuran (THF), 2-methyltetrahydrofuran, and combinations thereof. The polar protic solvent, the polar aprotic solvent, or a combination of the polar protic and polar aprotic solvent may be utilized with water or bine in liquid metal ion disinfecting composition. In one embodiment, the solvent comprises water. In another embodiment, the solvent comprises ethanol. In yet another embodiment, the solvent comprises water and a polar protic solvent such as ethanol or isopropanol. In yet another embodiment the solvent comprises water and a non-polar aprotic solvent such as DMSO. In still another embodiment, the solvent comprises water, a polar protic solvent such as ethanol, and a polar aprotic solvent such as acetonitrile.

Generally, the solvent can be present in an amount ranging from about 89.9 wt % to about 99.99 wt % based on the total weight of the liquid disinfectant composition. In various embodiments, the hydrophilic polymer can be present in an amount ranging from about 89.9 wt % to about 99.99 wt %, from about 90.0 wt % to about 99.9 wt %, from about 92.0 wt % to about 98.0 wt %, from about 93.0 wt % to about 97.0 wt %, or from about 94.0 wt % to about 96.0 wt % based on the total weight of the liquid disinfectant composition.

In general, the solvent may be present in an amount ranging from about 97.5 mole % to about 99.9 mole %. In various embodiments, the solvent may be present in an amount ranging from about 97.5 mole % to about 99.9 mole %, from about 98.0 mole % to about 99.5 mole %, or from about 98.5 mole % to about 99.0 mole %.

### (d) Chelating Agent, Surfactant, or a Combination of Chelating Agent and Surfactant

The liquid disinfectant composition may further include at least one chelating agent, at least one surfactant, or a combination of a chelating agent and a surfactant. The chelating agent and/or the surfactant interacts with the metal ion to form a complex which stabilizes the metal ion by additionally preventing moisture from interacting with the metal ion. This stabilization allows the liquid disinfectant composition to remain potent and active against a variety of pathogens.

The chelating agent is selected from a group consisting of citric acid, a citrate salt, tartaric acid, a salt of tartaric acid, ascorbic acid, an ascorbate salt, a polyaminocarboxylic acid, a salt of a polyaminocarboxylic acid, an organic compound, and combinations thereof.

In some embodiments, the chelating agent may be citric acid or a salt of citric acid. Non-limiting examples of salt of citric acid may be sodium citrate (also referred to as trisodium citrate), potassium citrate, ammonium citrate, magnesium citrate, and potassium magnesium citrate. In one embodiment, the chelating agent may be sodium citrate (trisodium citrate).

In other embodiments, the chelating agent may be ascorbic acid or an ascorbate salt. Non-limiting examples of suitable ascorbate salts may be sodium ascorbate, calcium ascorbate, ammonium ascorbate, and potassium ascorbate.

In other embodiments, the chelating agent may be tartaric acid or a salt of tartaric acid. Non-limiting examples of suitable salts of tartaric acid may be sodium tartrate, calcium tartrate, and ammonium tartrate.

In still other embodiments, the chelating agent may be a polyaminocarboxylic acid. Suitable non-limiting examples of polyaminocarboxylic acid may be iminodiacetic acid (IDA), nitrilotriacetic acid (NTA), ethylenediaminetetraacetic acid (EDTA), diethylenetriaminepentaacetic acid (DTPA), ethylene glycol-bis(p-aminoethyl ether)-N,N,N′,N′-tetraacetic acid (EGTA), 1,2-bis(o-aminophenoxy)ethane-N,N,N′,N′-tetraacetic acid (BAPTA), 1,4,7-triazacyclononane-1,4,7-triacetic acid (NOTA), 2,2′,2″,2′″-(1,4,7,10-tetraazacyclododecane-1,4,7,10-tetrayl)tetraacetic acid (DOTA), (2S)-1-[(3S)-3-{[(3S)-3-amino-3-carboxypropyl]amino}-3-carboxypropyl]azetidine carboxylic acid, ethylenediamine-N,N′-bis(2-hydroxyphenylacetic acid (EDDHA), ethylenediamine-N,N′-disuccinic acid (EDDS), and combinations thereof. The polyaminocarboxylic acid may be a free polyaminocarboxylic acid, a salt of the polyaminocarboxylic acid (polyaminocarboxylate), or a combination thereof. The polyaminocarboxylate salts may be an alkali metal salt, an alkali earth metal salt, or an organic salt. In one embodiment, the polyaminocarboxylic acid is ethylenediaminetetraacetic acid, a salt of an ethylenediaminetetraacetic acid, or a combination thereof. Suitable non-limiting examples of ethylenediaminetetraacetic acid salts may be monolithium ethylenediaminetetraacetic acid, disodium ethylenediaminetetraacetic acid, diammonium ethylenediaminetetraacetic acid, tetrasodium ethylenediaminetetraacetic acid, monocalcium ethylenediaminetetraacetic acid, and monobarium ethylenediaminetetraacetic acid. In one embodiment, the salt of the ethylenediaminetetraacetic acid is ethylenediaminetetraacetic acid.

In yet other embodiments, the chelating agent may be an organic compound. Suitable non-limiting examples of organic compounds may be group consisting of formic acid, glyoxilic acid, oxalic acid, acetic acid, glocolic acid, acrylic acid, pyruvic acid, malonic acid, propanoic acid, hydroxypropanoic acid, lactic acid, glyceric acid, fumaric acid, maleic acid, oxaloacetic acid, crotonoic acid, acetoacetic acid, 2-oxobutanoic acid, methylmalonic acid, succinic acid, methylsuccinic acid, malic acid, tartaric acid, dihydroxytartaric acid, butanoic acid, hydroxybutanoic acid, itaconic acid, mesaconic acid, oxoglutaric acid, glutaric acid, valeric acid, pivalic acid, aconitic acid, ascorbic acid, citric acid, isocitric acid, adipic acid, caproic acid, benzoic acid, salicylic acid, gentisic acid, protocatechuic acid, gallic acid, cyclohexanecarboxylic acid, pimelic acid, phthalic acid, terephthalic acid, phenylacetic acid, toluic acid, mandelic acid, suberic acid, octanoic acid, cinnamic acid, nonanoic acid, salts thereof, and combinations thereof.

The liquid disinfectant composition may also include a surfactant. The surfactant interacts with the metal ion, stabilizes the metal ion, and enhances the release of the metal cation from the salt in a solvent such as water or functions as a surfactant at interfaces between different components of the liquid disinfectant composition. The surfactant may be a cationic surfactant, an anionic surfactant, a nonionic surfactant, a zwitterionic surfactant, a nonionic surfactant, or a combination thereof.

Non-limiting examples of surfactants may be sulphonates, alkyl sulfates, alkylphenols, ethoxylated aliphatic alcohols, polyoxyethylenes, carboxylic esters, polyethylene glycol esters, fatty acid glycerol esters, fatty acid glycerol alcohols, quaternary ammonium salts, and so forth. In some embodiments, the surfactant is selected from a group consisting of benzalkonium chloride, cetalkonium chloride, cetrimonium bromide, cetrimonium chloride, sodium lauryl sulfate, sodium cocoyl isethionate, sodium dodecyl benzene sulfonate, sodium methyl oleoyl taurate, sodium lauryl sulfoacetate, sodium C₁₄₋₁₆ olefin sulfonate, disodium lauryl sulfosuccinate, cocamidopropyl betaine, lauramide MEA, sucrose stearate, cetyl alcohol, laureth-3, polysorbate-85, sorbitan monolaurate, PEG-30 castor oil, PEG-6 cocamide, distearyl dimethyl ammonium chloride, tetramethyl ammonium chloride, tetraethylammonium chloride, and combinations thereof. For example, silver nitrate is highly water soluble and would not require the use of a cationic surfactant to release the silver cation from the nitrate anion. In one embodiment, the surfactant is cetrimonium chloride. In another embodiment, the surfactant is sodium lauryl sulfate.

Silver chloride (AgCl) has a low water solubility and releases only a fraction of the silver cation. The use of the surfactant would enhance the release of more silver cation from the silver chloride.

In general, the weight ratio of the chelating agent, the surfactant, or a combination of chelating agent and surfactant, when present, may be present in an amount ranging from about 1.0 wt % to about 20.0 wt % based on the total weight of the liquid disinfectant composition. In various embodiments, the surfactant, or a combination of chelating agent and surfactant, when present, may be present in an amount ranging from about 1.0 wt % to about 20.0 wt %, from about 2.0 wt % to about 18.0 wt %, from about 5.0 wt % to about 15.0 wt %, from about 7.0 wt % to about 12.5 wt %, from about 8.0 wt % to about 12.0 wt %, or from about 9.0 wt % to about 11.0 wt % based on the total weight of the liquid disinfectant composition.

Generally, the weight ratio of the chelating agent, the surfactant, or a combination of chelating agent and surfactant, when present, to the metal salt ranges from about 100.0:1.0 to about 150.0:1.0. In various embodiments, the weight ratio of chelating agent, surfactant, or a combination of chelating agent and surfactant to metal salt ranges from about 100.0:1.0 to about 150.0:1.0, from about 110.0:1.0 to about 140.0:1.0, or from about 120.0:1.0 to about 130.0:1.0. In one embodiment, the weight ratio of the chelating agent, the surfactant, or a combination of chelating agent and surfactant to metal salt is about 125.0:1.0.

In general, the chelating agent, th surfactant, or a combination of one chelating agent and surfactant, when present, may be present in an amount ranging from about 0.1 mole % to about 1.0 mole %. In various embodiments, the chelating agent, the surfactant, or a combination of chelating agent and surfactant, when present, may be present in an amount ranging from about 0.1 mole % to about 1.0 mole %, from about 0.3 mole % to about 0.9 mole %, or from about 0.4 mole % to about 0.7 mole %.

### (e) Additives

The liquid disinfectant composition may optionally include at least one additive. The liquid disinfectant composition remains effective when the additive is not included in the composition. With the inclusion of the additive, a variety of disinfectant products can be produced and enhance the properties of the liquid disinfectant composition such as a synergistic effect. Non-limiting examples of these additives may be a wetting agent, a binding agent, an emulsifier, an essential oil, a protein material, or a combination thereof.

In some embodiments, the additive may be a wetting agent. The wetting agent may be selected from the group consisting of polyethoxylated castor oil; polypropylene glycol—polyethylene glycol block copolymers; polyoxyethylene sorbitan monooleate; sodium carboxymethyl cellulose; calcium carboxymethyl cellulose; hydrogenated or non-hydrogenated glycerolipids; ethoxylated or non-ethoxylated, linear or branched, saturated or monounsaturated or polyunsaturated C_{T} to C₃₀ fatty acids or salts thereof; cyclodextrin; alkaline earth metal or amine salts of ethoxylated or non-ethoxylated esters of sucrose; sorbitol; mannitol; glycerol or polyglycerol containing from 2 to 20 glycerol units; glycols combined with fatty acids, monoglycerides, diglycerides, triglycerides, or mixtures of glycerides of fatty acids; ethoxylated or non-ethoxylated, linear or branched, saturated or monounsaturated or polyunsaturated C₆ to C₃₀ fatty alcohols; sterols; cholesterol or derivatives thereof; ethoxylated or non-ethoxylated ethers of sucrose, sorbitol, mannitol, glycerol, or polyglycerol containing from 2 to 20 glycerol units; hydrogenated or non-hydrogenated, polyethoxylated vegetable oils; polyethylene glycol hydroxystearate; sphingolipids or sphingosine derivatives; polyalkyl glucosides; ceramides; polyethylene glycol-alkyl glycol copolymers; polyethylene glycol-polyalkylene glycol ether di-block or tri-block copolymers; diacetylated monoglycerides; diethylene glycol mono stearate; ethylene glycol monostearate; glyceryl monooleate; glyceryl monostearate; propylene glycol monostearate; polyethylene glycol stearate; polyethylene glycol ethers; polyethylene glycol hexadecyl ether; polyethylene glycol monododecyl ether; polyethylene glycol nonyl phenyl ethers; polyethylene glycol octyl phenyl ethers; octylphenoxy polyethoxyethanol; polyhydroxyethyl-tert-octylphenolformaldehyde; poloxamers; polysorbates; sorbitan monolaurate; sorbitan monooleate; sorbitan monopalmitate, sorbitan monostearate; sorbitan, sesquioleate; sorbitan trioleate; sorbitan tristearate; phospholipids; Kolliphor EL, Poloxamer 407, Tween 80, Triton X-100, macrogol stearate 400, macrogol stearate 2000, polyoxyethylene 50 stearate, macrogol ethers, cetomacrogol 1000, lauramacrogols, nonoxinols, octoxinols, tyloxapol, poloxamers, polysorbate 20, polysorbate 40, polysorbate 60, polysorbate 65, polysorbate 80, polysorbate 85, and combinations thereof.

In other embodiments, the additive may be a binding agent. The binding agent is selected from a group consisting of melamine, thiols, fatty acids, and combinations thereof.

In yet other embodiments, the additive is an essential oil or a derivative thereof. Non-limiting examples of suitable essential oils include (presented with scientific name of the plant from which it is derived and active ingredients): ajwain (Trachyspermum ammi; thymol), aniseed (Pimpinella anisum; α-pinene, camphene, β-pinene, linalool, cis-anethole, trans-anethole, safrole, anisaldehyde, acetoanisole), basil (Ocimum basilicum; linalol, methylchavikol, methylcinnamat, linolen), calamus (Acorus calamus; α-asarone, β-asarone, eugenol), capsicum (Capsicum annuum, Capsicum frutescen; capsaicin, capsaicinoids); caraway (Carum carvi; carvone, limonene, thymol, carvacrol, eugenol), cardamon (Elettaria cardomomum; α-pinene, β-pinene, sabinene, myrcene, α-phellandrene, limonene, 1,8-cineole, y-terpinene, p-cymene, terpinolene, linalool, linalyl acetate, terpinen-4-oil, α-terpineol, α-terpineol acetate, citronellol, nerol, geraniol, methyl eugenol, trans-nerolidol), chamomile (Matricaria; terpene bisabolol, farnesene, chamazulene, flavonoids (including apigenin, quercetin, patuletin and luteolin), coumarin), chervil (Anthriscus cerefolium; methyl chavicol), chrysanthemum (Chrysanthemum indicum; limonene, β-farnesene, 1,8-cineole, camphor, borneol, bornyl acetate), cinnamon (Cinnamomum zeylanicum; cinnamaldehyde, ethyl cinnamate, eugenol, beta-caryophyllene, linalool, methyl chavicol0, citron (Citrus sinensis fruit; limonene), clove (Syzygium aromaticum; eugenol, eugenyl acetate, caryophyllene), coriander (Coriandrum sativum; linalool, neryl acetate, γ-terpinene, α-pinene), dill (Anethum graveolens; d-carvone, dill apiol, eugenol, limonene, terpinene, myristicin), eucalyptus (Eucalyptus globulus; cineole, piperitone, phellandrene, citral, methyl cinnamate, geranyl acetate), garlic (Allium sativum; alliin, ajoene, diallyl polysulfides, vinyldithiins, S-allylcysteine), geranium (Rose Pelargonium x asperum; tannins such as gallic acid and flavone), gGinger (Zingiber officinale; (6)-gingerol, (6)-shagaol, (6)- and (10)-dehyro-gingerdione, (6)- and (10)-gingerdione, (6)-paradol, vallinoids, galanals A and B, zingerone), grapefruit (Citrus paradisi; α-pinene, sabinene, myrcene, limonene, geraniol, linalool, citronellal, decyl acetate, neryl acetate, terpinen-4-ol), honeysuckle (various varieties; linalool, ocimene, farnesene, germacrene D, eugenol, vanillin, (−)-methyl jasmonate, (+)-epi-methyl jasmonate, jasmone, (−)-jasmin lactone), juniper (Juniperus Communis; α-pinene, camphene, β-pinene, sabinene, myrcene, α-phellandrene, α-terpinene, ψ-terpinene, 1,4-cineole, β-phellandrene, p-cymene, terpinen-4-ol, bornyl acetate, cayophyllene, limonene, camphor, linalool, linalyl acetate, borneol, nerol), lavender, lemon (Citrus x limon; dl-limonene, α-pinene, l-α-terpineol, β-myrcene, β-pinene, β-linalool, α-terpinolene, terpinen-4-ol, cymene, E-citral). lemon balm (Melissa officinalis; eugenol, tannins, terpenes), lemongrass (Cymbopogon citratus; citral, myrcene, citronella, citronellol, geranilol), lime (Citrus aurantifolia, C. latifoli; d-limonene, beta-pinene, gamma-terpinene, citral), marjoram (Origanum majorana; thymol, sabinene, a terpinene, gamma terpinene, cymene, terpinolene, linalool, sabinene hydrate, linalyl acetate, terpineol, gamma terpineol), mint (Mentha spicata; menthol, menthone, menthyl acetate, menthofuran, 1,8-cineol), mustard oil (Brassica nigra, B. juncea; allylisothiocyanate, erucic acid, oleic acid, omega-3 alpha-linolenic acid, omega-6 linoleic acid), nutmeg (Myristica fragrans; myristicin, elemicin), oregano (Origanum vulgare; carvacrol, thymol, limonene, pinene, ocimene, caryophyllene), palmarosa (Cymbopogon martini; myrcene, linalool, geraniol, geranyl acetate, dipentene, limonene), peppermint (Mentha x piperita; menthol, menthone, menthyl acetate, menthofuran, 1,8-cineol), rose (Rosa damascena; citronellol, geraniol, nerol, linalool, phenyl ethyl alcohol, farnesol, stearoptene, α-pinene, β-pinene, α-terpinene, limonene, p-cymene, camphene, β-caryophyllene, neral, citronellyl acetate, geranyl acetate, neryl acetate, eugenol, methyl eugenol, rose oxide, α-damascenone, β-damascenone, benzaldehyde, benzyl alcohol, rhodinyl acetate, phenyl ethyl formate); rosemary (Rosmarinus officinalis; rosmarinic acid, camphor, caffeic acid, ursolic acid, betulinic acid, carnosic acid, carnosol), saffron (Crocus sativus; zeaxanthin, lycopene, α- and β-carotenes, picrocrocin, safranal, α-crocin), sage (Salvia officinalis; α-pinene, camphene, β-pinene, myrcene, limonene, 1,8-cineole, α-thujone, β-thujone, camphor, linalool, bornyl acetate, borneol), savory (Satureja montana, S. hortensis; carvarol, terpinene, paracymene, linalool, terpeneol, borneol), shiitake mushroom (Lentinula edodes; lentinan), tarragon (Artemisia dracunculus; methyl chavicol, methyl eugenol, trans-anethole, α-trans-ocimene, limonene, α-pinene, allo-ocimene, methyl eugenol, β-pinene, α-terpinolene), tea-tree oil (Melaleuca alternifolia; terpinen-4-ol, terpinolene, 1,8-cineole), thyme (Thymus vulgaris: thymol, p-cymene, myrcene, borneol, linalool), tumeric (Curcuma longa; curcumin, demethoxycurcumin, bisdemethoxycurcumin, turmerone, atlantone, zingiberene), and vetiver (Chrysopogon zizanioides; benzoic acid, vetiverol, furfurol, α and β-vetivone, vetivene, vetivenyl, vetivenate).

In still another embodiment, the additive is an emulsifier. The emulsifier and surfactant provide good foaming properties and improved viscosity which would be useful in hand soaps. Non-limiting examples of emulsifiers may be a dialkylamide or a monoalkylamide. In some embodiments, the emulsifier may be cocamide DEA.

In yet another embodiment, the additive is a protein material. This protein material allows the liquid disinfectant composition to be used on plant or animals. By way of non-limiting example, suitable plants include amaranth, arrowroot, barley, buckwheat, canola, cassava, channa (garbanzo), legumes, lentils, lupin, maize, millet, oat, pea, potato, rice, rye, sorghum, sunflower, tapioca, triticale, wheat, and mixtures thereof. The plant protein material may be canola meal, canola protein isolate, canola protein concentrate, maize or corn protein powder, maize or corn protein concentrate, maize or corn protein isolate, maize or corn germ, maize or corn gluten, maize or corn gluten meal, maize or corn flour, zein protein, glycoproteins, barley powder, barley protein concentrate, barley protein isolate, barley meal, barley flour, lupin flour, lupin protein isolate, lupin protein concentrate, oatmeal, oat flour, oat protein flour, oat protein isolate, oat protein concentrate, pea flour, pea protein isolate, pea protein concentrate, potato protein powder, potato protein isolate, potato protein concentrate, potato flour rice flour, rice meal, rice protein powder, rice protein isolate, rice protein concentrate, wheat protein powder, wheat gluten, wheat germ, wheat flour, wheat protein isolate, wheat protein concentrate, solubilized wheat proteins, or combinations thereof. In one embodiment, the protein material is zein protein.

Generally, the wetting agent, the binding agent, an essential oil, the emulsifier, the protein material, or a combination thereof, when present, may range from about 0.0 wt % to about 10 wt % of the total weight of the liquid disinfectant composition. In various embodiments, the wetting agent, the binding agent, an essential oil, the protein material, or a combination thereof, when present, may range from about 0.0 wt % to about 10.0 wt %, from about 0.1 wt. % to about 8.0 wt. %, from about 0.5 wt % to about 4.0 wt %, or from about 1.0 wt % to about 2.5 wt % of the total weight of the liquid disinfectant composition. In one embodiment, the wetting agent, the binding agent, an essential oil, the emulsifier, the protein material, or a combination thereof, when present, may be about 0.0 wt % of the total weight of the liquid disinfectant composition. In another embodiment, the wetting agent, the binding agent, an essential oil, the emulsifier, the protein material, or a combination thereof, when present, may be about 1.0 wt % of the total weight of the liquid disinfectant composition. In yet another embodiment, the wetting agent, the binding agent, an essential oil, the emulsifier, the protein material, or a combination thereof, when present, may be about 6.0 wt % of the total weight of the liquid disinfectant composition.

### (f) Preferred Embodiments

In some embodiments, the solvent is water and is present in an amount of about 90.0 wt % to about 99.9 wt %; the metal salt is a silver salt, a copper salt, a zinc salt, a combination of a silver salt and a copper salt, a combination of a silver salt and a zinc salt, or combination of a silver salt, a copper salt, and a zinc salt and is present in an amount of between about 0.001 wt % to about 5.0 wt %; the hydrophilic polymer is either PVP K-30 or PVP K-90 and present in an amount of between about 0.1 wt % to about 5.0 wt %. The chelating agent, the surfactant, or a combination of chelating agent and surfactant is ethylenediaminetetraacetic acid, sodium citrate, sodium lauryl sulfate, centrimonium chloride, or combinations thereof, and is present in an amount between about 0.1 wt % to about 20 wt %.

In certain embodiments, the solvent is water and is present in an amount between about 90.0 wt % to about 99.9 wt %; the metal salt is a silver salt, namely silver nitrate, and is present in an amount between about 0.01 wt % to about 1.0 wt %; the hydrophilic polymer is either PVP K-30 or PVP K-90 and present in an amount between about 0.1 wt % to about 5.0 wt %. The chelating agent, the surfactant, or a combination of chelating agent and surfactant is ethylenediaminetetraacetic acid, and is present in an amount of between about 0.1 wt % to about 10 wt %.

In certain embodiments, the solvent is water and is present in an amount of between about 90.0 wt % to about 99.9 wt %; the metal salt is a silver salt, namely silver nitrate, and is present in an amount of between about 0.01 wt % to about 1.0 wt %; the hydrophilic polymer is either PVP K-30 and present in an amount of between about 0.1 wt % to about 5.0 wt %. The chelating agent, the surfactant, or a combination of chelating agent and surfactant is trisodium citrate and is present in an amount of between about 0.1 wt % to about 10 wt %.

In certain embodiments, the solvent is water and is present in an amount of between about 90.0 wt % to about 99.9 wt %; the metal salt is a silver salt, namely silver chloride, and is present in an amount of between about 0.01 wt % to about 1.0 wt %; the hydrophilic polymer is PVP K-30 and is present in an amount of between about 0.1 wt % to about 5.0 wt %. The chelating agent, the surfactant, or a combination of chelating agent and surfactant is cetrimonium chloride and is present in an amount of between about 0.1 wt % to about 10 wt %.

In certain embodiments, the solvent is water and is present in an amount of between about 90.0 wt % to about 99.9 wt %; the metal salt is a silver salt, namely silver chloride, and is present in an amount of between about 0.01 wt % to about 1.0 wt %; the hydrophilic polymer is PVP K-30 and is present in an amount of between about 0.1 wt % to about 5.0 wt %. The chelating agent, the surfactant, or a combination of chelating agent and surfactant is sodium lauryl sulfate and is present in an amount of between about 0.1 wt % to about 10 wt %.

In certain embodiments, the solvent is water and is present in an amount of between about 90.0 wt % to about 99.9 wt %; the metal salt is silver acetate, copper acetate, zinc acetate, a combination of silver acetate and copper acetate, a combination of silver acetate and zinc acetate, a combination of copper acetate and zinc acetate, or a combination of silver acetate, copper acetate, and zinc acetate and is present in an amount of between about 0.01 wt % to about 1.0 wt %; the hydrophilic polymer is PVP K-30 and is present in an amount of between about 0.1 wt % to about 5.0 wt %.

### (g) Properties of the Liquid Disinfectant Composition

The liquid disinfectant composition, as disclosed herein, has many unique properties.

The liquid disinfectant compositions are light stable. Light stability enables the liquid disinfectant composition to be applied to articles placed in direct sunlight and maintain the disinfectant properties for up to 60 days. The liquid disinfectant composition exhibits antimicrobial properties, antibacterial properties, antiviral properties, antifungal properties, or a combination thereof against a variety of pathogens as verified by the following tests: for bacteria and fungi: AOAC Use Dilution Method (UDM), ASTM E 2315, ISO 22196:2011; and for viruses: AATCC 100-20124, ISO18184:2019, ISO 21702:2019, Rt-PCR, liquid-liquid contact. For viruses, the Fonsum Pharma test against a Covid-19 RT-PCR Evaluation was utilized. The pathogen kill rate is greater than 99% after less than a 5-minute period of time and pathogenic sterility is maintained for up to 60 days. In some embodiments, pathogenic sterility is maintained on a surface for up to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days. Light stability also enables the liquid disinfectant composition, at various concentrations, to be stored in light without reduction in the efficacy of the composition.

The liquid disinfectant compositions are heat stable. Heat stability allows the liquid disinfectant composition to be applied to articles above room temperature and maintain the disinfectant properties for up to 60 days. The liquid disinfectant composition exhibits antimicrobial properties, antibacterial properties, antiviral properties, antifungal properties, or a combination thereof against a variety of pathogens as verified by the following tests: for bacteria and fungi: AOAC Use Dilution Method (UDM), ASTM E 2315, ISO 22196:2011; and for viruses: AATCC 100-20124, ISO18184:2019, ISO 21702:2019, Rt-PCR, liquid-liquid contact. For viruses, the Fonsum Pharma test against a Covid-19 RT-PCR Evaluation was utilized. The pathogen kill rate is greater than 99% after less than a 5-minute period of time and pathogenic sterility is maintained for up to 60 days. In some embodiments, pathogenic sterility is maintained on a surface for up to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

The liquid disinfectant compositions, as disclosed herein, do not contain nanoparticles. Examples of the liquid disinfectant composition have been evaluated by ultraviolet (UV)-visible spectroscopy. Nanoparticles have unique optical properties that are sensitive to the size, shape, concentration, agglomeration state, and refractive index near the nanoparticle surface, which makes UV-Vis a valuable tool for identifying, characterizing, and studying nanomaterials. Generally, nanoparticles provide colored solutions.

Since nanoparticles are considered toxic, the presence of such nanoparticles would render the liquid disinfectant toxic. Nanoparticles have the ability to cross biological membranes and access cells, tissues and organs that larger-sized particles which normally cannot. Once nanoparticles gain access to the blood stream via inhalation, ingestion, or through a cut, the nanoparticles might lead to both genotoxicity and biochemical toxicity. Also, once the nanoparticles gain access to the xylem and phloem of a plant, the nanoparticles can provide some positive attributes such as accelerated growth, enhanced yield, lower use of fertilizer, etc. as well as remain present in the plant. In addition, use of nanoparticles also affects soil health, environmental quality, aquatic life, and animal health. Moreover, the accelerated use of nanomaterials has greatly raised the concerns about the toxicity in food safety and ecosystem. In order to verify that the liquid disinfectant compositions do not contain nanoparticles, as disclosed herein, the compositions were evaluated by an analytical method, such as UV-vis spectrometry. Since the liquid disinfectant composition does not contain nanoparticles, these liquid disinfectant compositions are considered non-toxic.

The liquid disinfectant composition has a pH that ranges from about 6 to about 8. As such, these compositions are considered neutral and non-corrosive. Given its neutral and non-corrosive properties, the liquid disinfectant composition can be used on various articles and surfaces without causing the article or surface to deteriorate or decompose (e.g., such as an iron surface which would rust in the presence of other disinfecting compositions). The liquid disinfectant composition of the present disclosure may additionally be applied to skin, animals, fruits, vegetables, or plants without any harmful side effects.

The liquid disinfectant composition exhibits antimicrobial properties, antibacterial properties, antiviral properties, antifungal properties, or a combination thereof against a variety of pathogens as verified by the following tests: for bacteria and fungi: AOAC Use Dilution Method (UDM), ASTM E 2315, ISO 22196:2011; and for viruses: AATCC 100-20124, ISO18184:2019, ISO 21702:2019, Rt-PCR, liquid-liquid contact. For viruses, the Fonsum Pharma test against a Covid-19 RT-PCR Evaluation was utilized. The pathogen kill rate is greater than 99% after less than a 5-minute period of time and pathogenic sterility is maintained for up to 60 days. In some embodiments, pathogenic sterility is maintained on a surface for up to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

The liquid disinfectant composition may be prepared to concentrations of 100 parts per million (ppm) or more. Generally, a concentration of 100 ppm or less is adequate to provide a highly effective disinfectant. Even after 28 days, these solutions maintain potency, can effectively disinfect various surfaces, and remain colorless. At higher concentrations, the liquid disinfectant composition may produce a color after a few days. Generally, greater than 100 ppm concentration of silver will be stored in the dark. Even though the color is not pleasing to a customer, the color is an indication that a small amount of silver oxide species is present. Yet, the colored solution will maintain the disinfectant properties.

The liquid disinfectant composition is highly durable, meaning that after the composition has been applied to a surface or article, the surface or article may be wiped numerous times without removing or reducing the efficacy of the composition. After an initial coating of the liquid disinfectant composition has been applied to a surface or article, the surface or article may be wiped (scoured) more than 160 times without pathogen regrowth.

### (II) Methods of Preparing Liquid Disinfectant Compositions

In another aspect, the present disclosure provides method of preparing a liquid disinfectant composition. The method comprises: contacting a metal salt in a solvent to form a mixture; and (b) contacting the mixture from step (a) with a hydrophilic polymer; wherein the liquid disinfectant composition does not comprise nanoparticles; and wherein the hydrophilic polymer prevents oxidation of the metal ion and maintains contact with a variety of surfaces. The method further comprises contacting a chelating agent, a surfactant, or a combination of chelating agent and surfactant in step (a). The method, as described above, is economical, easily performed, scalable, and produces a highly effective liquid disinfectant composition.

The methods, as disclosed herein, may be conducted in batch, semi batch, or a continuous mode. The methods may be conducted in the dark and/or under an inert atmosphere and are not necessarily required to prepare the liquid disinfectant composition.

(a) Contacting at Least One Metal Salt with at Least One Solvent to Form a Mixture

The first step in the method comprises contacting a metal salt with a solvent to form a mixture. Suitable metal salts and solvents are described in more detail above in Section (I). In one embodiment, the metal salt is silver nitrate. In another embodiment, the metal salt is silver chloride. In yet another embodiment, the suitable solvent in step (a) is water.

Step (a) may be conducted under an inert atmosphere. Suitable inert gases may be helium, nitrogen, argon, or a combination thereof.

In step (a), the metal salt may be added portion-wise or entirely to the solvent upon stirring to form a mixture. Suitable methods are known in the art for stirring mixtures of solids, such as magnetic stirring, mechanical stirring, jet mixers, etc.

The temperature of mixing in step (a) may range from about 0° C. to about 50° C. In various embodiments, the temperature of mixing in step (a) ranges from about 0° C. to about 50° C., from about 10° C. to about 35° C., or from about 20° C. to about 30° C. In an embodiment, the temperature of mixing in step (a) is about 23° C. (room or ambient temperature).

The duration of mixing ranges from about 1 minute to about 30 minutes until a homogeneous solution is obtained by visual determination. In various embodiments, the duration of mixing ranges from about 1 minute to about 30 minutes, from about 2 minutes to about 15 minutes, or from about 3 minutes to about 10 minutes until a homogeneous solution is obtained as visually determined.

(b) Contacting the Mixture from Step (a) with at Least One Hydrophilic Polymer Forming the Liquid Disinfectant Composition

The next step in the method comprises contacting the mixture from step (a) with a hydrophilic polymer. A list of suitable hydrophilic polymers is described in more detail above. The hydrophilic polymer may be added in portions or all at once. In one embodiment, the hydrophilic polymer is PVP K-30. In another embodiment, the hydrophilic polymer is PVP K-90. After the hydrophilic polymer is incorporated into the mixture from step (a), the liquid disinfectant composition is prepared.

The temperature in step (b) ranges from about 0° C. to about 50° C. In various embodiments, the temperature in step (b) ranges from about 0° C. to about 50° C., from about 10° C. to about 35° C., or from about 20° C. to about 30° C. In an embodiment, the temperature is about 23° C. (room temperature).

The duration of step (b) ranges from about 1 minute to about 60 minutes. In various embodiments, the duration of step (b) ranges from about 1 minute to about 60 minutes, from about 5 minutes to about 30 minutes, or from about 10 minutes to about 20 minutes, or about 5 minutes.

The method further comprises contacting a chelating agent, a surfactant, or a combination of chelating agent and surfactant in step (a). Suitable chelating agents and surfactants are detailed above. In some embodiments, the suitable chelating agent is a polyaminocarboxylic acid or a salt of a polyaminocarboxylic acid. In other embodiments, the suitable chelating agent is sodium citrate (trisodium citrate). In one embodiment, the polyaminocarboxylic acid is ethylenediaminetetraacetic acid. In another embodiments, the chelating agent is sodium citrate (trisodium citrate). In one embodiment, the surfactant is sodium lauryl sulfate. In another embodiment, the surfactant is cetrimonium chloride.

After the method is completed, the liquid disinfectant composition is prepared. The liquid disinfectant composition may be evaporated producing a solid, such as a white powder. The solvent in the liquid disinfectant may be evaporated under reduced pressure (vacuum) and/or an inert atmosphere producing the solid. Other non-limiting methods of removing the solvent from the disinfectant composition may be spray drying or lyophilization. Thus, the disinfectant composition may be in the form of a liquid or solid.

The liquid disinfectant composition may be included in a gel or a foam comprising viscosity adjusting additives, an emulsion, or into a solid. When a gel form is desired, a viscosity-adjusting additive may be employed (e.g., carbomers, cellulose derivatives, or other gelling agents).

Since the liquid disinfectant composition is light stable, heat stable, non-corrosive, and non-toxic, the disinfectant composition may be infused or included into a variety of products such as hand sanitizers, soaps, detergents, fabric softeners, cleaners, plant fertilizers, pesticides, insecticides, herbicides, insect repellents, paints, varnishes, adhesives, sealants, glass, grout, plastics, thermoplastics, wood, cardboard, cement, health and hygiene products, dairy products (such as milk, butter, and cheese), animal feed, and pet feed. With the inclusion or infusion of the liquid disinfectant composition as a liquid or a powder into these products, these products perform their entitled purpose but also provide disinfectant properties to these products.

The yield of the liquid disinfectant composition from the method may be greater than 95% or greater than 99%.

### (III) Methods for Disinfecting a Surface or an Article

In another aspect, the present disclosure provides methods of disinfecting and/or maintaining the pathogenic sterility of an article and a method of cleaning the surface of an article. The method comprises contacting the surface of the article with the liquid disinfectant composition or including the liquid disinfectant composition within the article.

(a) Liquid Disinfectant Compositions

The liquid disinfectant compositions are described in more detail above in Section (I).

(b) Surfaces or Articles

The liquid disinfectant composition may be applied to various surfaces or articles. The surfaces or articles may be made from a variety of materials which may be porous or non-porous. The articles may be made from a variety of materials and such as but not limited to latex, paper, cloth, and plastic. Non-limiting examples of these surfaces may be metals or metal alloys (for example, steel, stainless steel, iron), wood, cardboard. glass, plastic, thermoplastic, ceramic, natural stone (for example, granite, marble, quartz, quartzite), synthetic stone, concrete, sheet rock, livestock living spaces (such as a barn, coup, a stable, and the like), fruits, vegetables, eggs, seeds, raw meat surfaces, and the like. Non-limiting examples of these articles may be dairy products, animal feed, pet feed, water, and the like. By applying the liquid disinfectant composition, these surfaces and articles would be disinfected, preserved, and sterilized.

The surface or article may be located in a hospital or a doctor's office and used for health care. The liquid disinfectant composition may be applied to, for instance, a catheter, furniture, floors, linens, drapes, wheelchairs, walkers, and the like in order to disinfect surfaces within a hospital. The surfaces will remain disinfected for up to 60 days, even after numerous touches by a human. In some embodiments, the surfaces will remain disinfected for up to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

The liquid disinfectant composition may be used in health care and may be used either in-vivo or in-vitro. Non-limiting examples of in-vitro uses may be sterilization of medical surgical equipment or surgical instruments (such as a probe forceps, respirators, etc.), disinfection of hands and/or extremities such as a surgical handwash or surgical scrub; wound care, and plasma preservation. Non-limiting examples of in-vivo uses may medical textiles (such as gauze, bandages, etc.), nasal sprays, irrigation solutions, tablet coatings, medical implants or devices, and dental uses such as dental crowns, dental implants, etc. The disinfecting composition may be applied directly to a wound or an incision the covered by a bandage; or applied to a bandage or gauze then directly applied to a wound or incision. This application would reduce the time for healing of the wound or incision.

The surface or the article may be personal protective equipment (PPE). The disinfectant composition may be applied as a liquid or a solid to the internal surface or external surface of a face mask or respirator, gloves, mask, and aprons.

The article may be an air filter. The liquid or powder disinfectant composition may be applied to the internal surface or external surface air filter.

The article may be in a home, a housing structure, or a building. Suitable, non-limiting examples of these articles may be a wood table, a counter surface (Formica, stainless steel, quartz, granite, etc.), a faucet (stainless steel, chromed steel), a shower head, a floor (such as a bathroom floor), tiles, sinks, showers, toilets, tubs, railings, door handles, doors, dishwashing machines, cloths driers, etc. After the article is treated with the disinfectant composition, these articles will remain disinfected for up to 60 days. In some embodiments, the surfaces will remain disinfected for up to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

The article may be a food container, food packaging, or a food preservative. The liquid disinfectant composition as a liquid or a powder may be applied directly to a food container or food packaging material to prevent microbial growth and extend the freshness of the food such as meat, poultry, eggs, and cheese. Suitable, non-limiting examples of food containers or food packaging may be plastic wrap, aluminum foil, a stainless-steel container, plastic containers, glass containers, plastic deli containers, etc. The liquid disinfectant composition may be directly applied to the external surface of fresh meat or fresh seafood such as goat, beef, chicken, pork, turkey, duck, lobsters, fish, and alike. By applying the liquid disinfectant composition, the pathogens present on the surface will be eliminated. As a food preservative or in can preservative, the liquid disinfectant composition as a liquid or a powder may be sprayed or included before canning or bottling of a meat, fruit, or vegetable, or as an additive after the meat, fruit, or vegetable is introduced into the can or bottle.

The article may be a building material. After applying the disinfectant composition, the building material can easily be used without the fear of mold or bacteria growth in the future. Suitable, non-limiting examples may be wood, paper, sheet rock, iron, wall paper, stainless steel, etc.

The article may be water. The addition of the liquid disinfectant composition as a liquid or a powder would aid in the potability of water and use in sanitation. The addition of a few drops of the liquid disinfection solution or a small amount of the powder disinfectant composition would kill the pathogens and make the water suitable to drink or for use in washing.

The article may be a polymer, a thermoplastic, or a plastic. The liquid disinfectant composition may be added before the polymer, the thermoplastic, or the plastic as the polymer, the thermoplastic, or the plastic is produced (in the production process) or after the polymer, the thermoplastic, or the plastic is produced. Suitable, non-limiting examples may be a toy, a polymer coated counter surface, a plastic item, a toy, a plastic film, etc.

The article may be an article already effected by bacteria or mold. By applying the liquid metal disinfectant composition, the bacteria or mold would be eliminated, and the article could be reused. Suitable, non-limiting examples may be a moldy bathroom wall, moldy sheet rock, a moldy bathroom floor, a moldy pipe, etc.

The disinfectant composition may be added to or applied to paint, caulk, varnish, and concrete. The paint, caulk, varnish, and concrete would not only eliminate pathogens already present on the surface of the surface or article but also prevent pathogens from growing in the future.

(c) Applying the Liquid Disinfectant Composition

The liquid disinfectant composition as a liquid or a powder may be applied in various methods. The liquid disinfectant composition may be rapidly sprayed or cast in thin layers over large areas or sprayed and coated numerous times on the surface or article.

In order to identify whether the surface or article has been fully coated, a simple touch test with a finger, a corner of a towel, etc. can be utilized. If a portion of the article or the surface has not been coated, an additional application of the disinfectant composition may be applied to ensure full and complete coverage of the surface or article. The liquid disinfectant composition may be applied in an aqueous solution to an article or on a surface. Various coating techniques include, but are not limited to, spray coating, dip coating, doctor-blade coating, spin coating, air knife coating, single and multilayer slide coating, gap coating, knife-over-roll coating, metering rod (Meyer bar) coating, reverse roll coating, rotary screen coating, extrusion coating, casting, using a paint brush, wiping, or printing. The composition may be rapidly sprayed or cast in thin layers over large areas or sprayed and coated numerous times on the surface or article.

As a solid, the disinfectant composition may be applied in various methods. Non-limiting methods of applying a solid are dry spraying, rolled, or cast.

(d) Properties of Articles after Contacting with the Liquid Disinfectant Composition

The surfaces or articles, after being disinfected by the liquid disinfectant composition, would kill greater than 99% of pathogens present as compared to articles that have not been treated with the liquid disinfectant composition. With the durability, the non-corrosive, and non-toxic nature of the disinfecting composition, the treated articles may be touched or contacted with the skin (such as fingers, arms, hands, etc.) for more than 100 times up to a 60-day period. In some embodiments, the surfaces may be touched or contacted with the skin more than 100 times up to a 30-day period. In other embodiments, the surfaces may be touched or contacted with the skin more than 100 times up to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days. Even after this repeated contact, no growth of pathogens was detected.

### Definitions

Unless defined otherwise, all technical and scientific terms used herein have the meaning commonly understood by a person skilled in the art to which this invention belongs.

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention.

Reference throughout this specification to “one embodiment,” “some embodiments”, “certain embodiments,” “one or more embodiments,” or “an embodiment” means that a particular feature, structure, material, or characteristic described in connection with the embodiment is included in at least one embodiment of the invention. Thus, the appearances of phrases containing the term “embodiment(s)” in various places throughout this specification are not necessarily referring to the same embodiment of the invention. Furthermore, the particular features, structures, materials, or characteristics may be combined in any suitable manner in one or more embodiments.

In the present disclosure, “%” refers to “weight % (wt. %)” or “mass %”, unless otherwise stated.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

As used herein, the phrase “consisting of excludes any element, step, or ingredient not specified in the claim. When the phrase “consists of (or variations thereof) appears in a clause of the body of a claim, rather than immediately following the preamble, it limits only the element set forth in that clause; other elements are not excluded from the claim as a whole. As used herein, the phrase “consisting essentially of limits the scope of a claim to the specified elements or method steps, plus those that do not materially affect the basis and novel characteristic(s) of the claimed subject matter.

When introducing elements of the embodiments described herein, the articles “a”, “an”, “the” and “said” are intended to mean that there are one or more of the elements.

The terms “comprises”, “comprising”, or any other variations thereof used in the disclosure, are intended to cover a non-exclusive inclusion, such that a device, apparatus, system, assembly, method that comprises a list of components or a series of steps that does not include only those components or steps but may include other components or steps not expressly listed or inherent to such apparatus, or assembly, or device. In other words, one or more elements or steps in a system or device or process proceeded by “comprises . . . a” or “comprising . . . of does not, without more constraints, preclude the existence of other elements or additional elements or additional steps in the system, device, or process as the case may be. Besides, the use of “comprising”, “consisting” or “including” also contemplates embodiments that “consist essentially of or “consist of the recited formulation and steps of preparation of the formulation.

As used herein, the term “powder,” in all of its forms, refers to a dry, bulk solid composed of a multitude of fine particles, such as finely dispersed solid particles. The powder may be characterized by an average particle size of from about 1.0 micron to about 1000 microns, or from about 1.0 micron to 100 microns.

As used herein, the term “nanoparticle,” in all of its forms, refers to a particle characterized by a particle size of less than one micron. The use of the term in this application refers to particles having a size that are not desirous in the composition because they may be toxic to the user.

As used herein, the term “light stable”, in all of its forms, refers to the disinfectant composition not losing efficacy or potency in the presence of light either sunlight or manmade light.

As various changes could be made in the above-described methods without departing from the scope of the invention, it is intended that all matter contained in the above description and in the examples given below, shall be interpreted as illustrative and not in a limiting sense.

### EXAMPLES

While the present invention is disclosed in reference to the preferred embodiments or examples above, it is to be understood that these embodiments or examples are intended for illustrative purposes, which shall not be treated as limitations to the present invention. It is contemplated that modifications and combinations will readily occur to those skilled in the art, which modifications and combinations will be within the spirit of the invention and the scope of the following claims.

Materials and Instruments:

The following materials were sourced in the Examples noted below: Ethylenediaminetetraacetic acid (EDTA), trisodium citrate, sodium lauryl sulfate, cocamide DEA, and zein powder were sourced from Analab Fine Chemicals, Gujarat, India or Sigma Aldrich and used without further purification. The purity of these reagents was greater than 99%. Silver nitrate, silver chloride, copper (II) acetate, zinc acetate, zinc citrate, and silver lactate were sourced from Rochester Silver, Rochester, N.Y., Alpha Chemika, or Sigma Aldrich and used without further purification. The minimum assay of these reagents was 99% minimum. Polyvinylpyrrolidone K-30 (PVP K-30) and polyvinylpyrrolidone K-90 (PVP K-90) was sourced from Alpha Chemika and used directly without further purification.

The pH of the disinfectant composition was determined using a Systonic digital auto pH meter with Combination pH Electrode calibrated with a pH 7.0 buffer. The concentration of silver ions in the samples was determined by an inductively coupled plasma optical emission spectrometry (ICP-OES) method or potentiometric titration using 1 drop nitric acid and titrating with 100 ppm solution of sodium chloride. The presence of nanoparticles was determine using UV-vis spectroscopy.

### Example 1: General Procedure for Preparation of Light Stable Liquid Disinfectant Compositions

Specific amounts of reagents used in the general preparation of the liquid disinfectant composition are shown below in the following tables shown below.

The chelating agent, the surfactant, or a combination of the chelating agent and surfactant, and water were placed into a flask equipped with magnetic stirring at room temperature. After a colorless solution was obtained, the water-soluble metal salt was added to the chelating agent, the surfactant, or a combination of the chelating agent and surfactant and water. The mixture was stirred for 5 minutes at room temperature until the mixture appeared to be colorless. Then, the hydrophilic polymer was added portion wise into the water-soluble metal salt, the chelating agent, the surfactant, or a combination of the chelating agent and surfactant and water. After the hydrophilic polymer was added, this mixture was stirred for an additional 5 minutes until a colorless mixture appeared. A 0.5 mL portion of the liquid was removed and tested for the pH. The pH of the solution was measured using a pH meter resulting in a pH of approximately 6.5-7.0. Yield: 99%. The light stable liquid disinfectant composition was stored in a plastic bottle and stored at ambient temperatures.

This general example demonstrates that the order of addition is important to prepare the liquid disinfectant composition as a colorless solution.

### Example 2: Preparation of Light Stable Disinfectant Compositions Using Silver Nitrate, EDTA, and PVP-K30 and PVP-K90

The procedure for preparing the light stable disinfectant composition using silver nitrate, EDTA, and PVP K-30; and silver nitrate, EDTA, and PVP K-90 is described in more detail in the general procedure in Example 1. Table 1 indicates the amounts of reagents used in the preparation of the silver light stable disinfectant solution using PVP K-30. Table 2 indicates the amount of indicates the amounts of reagents used in the preparation of the silver light stable disinfectant solution using PVP K-90.

TABLE 1
Light Stable Silver Disinfectant Compositions using PVP K30

| | | Amount | Amount | Amount | | Concentration |
|---|---|---|---|---|---|---|
| Experiment | Formulation | EDTA | AgNO₃ | PVP K- | Water | of Silver |
| # | # | (g) | (g) | 30 (g) | (mL) | (ppm) |
| 1 | 1 | 1.25 | 0.01 | 0.5 | 100 | 100 |
| 2 | 2 | 1.25 | 0.02 | 0.5 | 100 | 200 |
| 3 | 3 | 1.25 | 0.03 | 0.5 | 100 | 300 |
| 4 | 4 | 1.25 | 0.04 | 0.5 | 100 | 400 |
| 5 | 5 | 1.25 | 0.05 | 0.5 | 100 | 500 |
| 6 | 6 | 6.25 | 0.05 | 2.5 | 100 | 500 |
| 7 | 7 | 10.0 | 0.08 | 4.0 | 100 | 800 |
| 8 | 8 | 1.25 | 0.01 | 1.0 | 100 | 100 |
| 9 | 9 | 2.5 | 0.02 | 2.0 | 100 | 200 |
| 10 | 10 | 2.5 | 0.02 | 2.0 | 100 | 200 |
| 11 | 11 | 1.25 | 0.01 | 0.5 | 100 | 100 |
| 12 | 12 | 1.25 | 0.05 | 0.5 | 100 | 500 |
| 13 | 13 | 2.5 | 0.1 | 1.0 | 500 | 200 |
| 14 | 14 | 9.0 | 0.01 | 2.0 | 100 | 1000 |
| 15 | 15 | 1.25 | 0.05 | 0.5 | 500 | 100 |
| 16 | 16 | 2.5 | 0.01 | 1.0 | 500 | 200 |
| 17 | 17 | 1.25 | 0.01 | 2.0 | 100 | 100 |
| 18 | 18 | 2.5 | 0.01 | 2.0 | 100 | 100 |
| 19 | 19 | 2.5 | 0.02 | 2.0 | 100 | 200 |
| 20 | 20 | 1.25 | 0.01 | 2.0 | 100 | 100 |
| 21 | 21 | 1.0 | 2.0 | 4.0 | 100 | 20000 |
| 22 | 22 | 2.0 | 1.0 | 4.0 | 100 | 10000 |
| 23 | 23 | 2.0 | 1.0 | 4.0 | 100 | 10000 |
| 24 | 24 | 2.0 | 1.0 | 4.0 | 100 | 10000 |
| 25 | 25 | 10.0 | 0.08 | 4.0 | 100 | 800 |
| 26 | 26 | 2.5 | 0.02 | 1.0 | 100 | 200 |
| 27 | 27 | 2.5 | 0.02 | 1.0 | 100 | 200 |
| 28 | 28 | 2.5 | 0.02 | 1.0 | 100 | 200 |

EDTA: ethylenediaminetetraacetic acid.
PVP K-30: Polyvinyl pyrrolidine K-30;
Concentration of Silver was determined through potentiometric titration.

TABLE 2
Light Stable Silver Compositions using PVP K-90

| | | Amount | Amount | Amount | | |
|---|---|---|---|---|---|---|
| Experiment | Formulation | EDTA | AgNO₃ | PVP K-90 | Water | Concentration |
| # | # | (g) | (g) | (g) | (mL) | of Silver (ppm) |
| 1 | 54 | 1.25 | 0.01 | 2.0 | 100 | 100 |
| 2 | 55 | 1.25 | 0.02 | 2.0 | 100 | 200 |
| 3 | 56 | 1.25 | 0.03 | 2.0 | 100 | 300 |
| 4 | 57 | 1.0 | 0.03 | 2.0 | 100 | 300 |

EDTA: ethylenediaminetetraacetic acid.
PVP K-90: Polyvinyl pyrrolidine K-90;
Concentration of silver was determined through potentiometric titration as described above.

### Example 3: ASTM E-2315 Test to Assess the In Vitro Reduction of a Microbial Population of Test Organisms after Exposure to the Liquid Disinfectant Compositions

An ASTM E-2315 was conducted under guidelines of the AOAC (Association of Official Analytical Chemists). A pure culture of Escherichia Coli (E. Coli, ATCC 25922) was streaked on Soyabean Casein Digest Agar plates and allowed to incubate at 37° C. for up to 2 days. Following incubation, the surface of agar plate was scraped, and the growth suspension was adjusted to a concentration of 106 cfu/ml. Test and control substances were dispensed in identical volumes to sterile test tubes. Independently, test and control substances were inoculated with the test microorganism and mixed. Control suspensions were immediately plated to represent the concentration present at the start of the test or time zero and at the conclusion of each contact time; a volume of the liquid test solution was neutralized. Dilutions of the neutralized test solution were placed on to appropriate agar plates and incubation temperatures to determine the surviving microorganisms at the respective contact times and reductions of microorganisms were calculated by comparing initial microbial concentrations to surviving microbial concentrations. The samples showed greater than 99.99% reduction on exposure to Escherichia coli when exposed for just 15 seconds, thereby demonstrating instant killing activity of the composition as compared to the control. This data is presented in Table 3 for the disinfectant compositions shown in Tables 1 and 2 shown above. Similar tests were conducted Staphylococcus aureus (ATCC 25923) and Pseudomonas aeruginosa (ATCC 9027) showing the same instant kill rate of the composition as compared to the control.

TABLE 3
Experimental Results for ASTM E-2315 Evaluation

| | | | | Pseudomonas | |
|---|---|---|---|---|---|
| | | S. aureus | E. coli | aeruginosa | |
| | | Reduction | Reduction | Reduction | |
| Experiment | Formulation | (exposure | (exposure | (exposure | Exposure |
| # | # | time 5 min) | time 5 min) | time 5 min) | Time |
| 1 | 2 | 99.99999 | 99.999999 | 99.999999 | 5 min |
| 2 | 1 | 99.99999 | 99.999999 | 99.999999 | 5 min |
| 4 | 5 | 99.999999 | | 99.99 | 5 min |
| 4 | 5 | 99.999999 | | 99.999 | 10 min |
| 5 | 13 | 99.999999 | | 99.99 | 5 min |
| 5 | 13 | 99.999999 | | 99.999 | 10 min |
| 8 | 18 | | 99.999999 | 99.999999 | 5 min |
| 9 | 27 | | | 99.9 | 5 min |
| 10 | 18 | | 99.999999 | | 5 min |
| 11 | 20 | | 99.999999 | | 5 min |
| 12 | 19 | | 99.999999 | | 5 min |
| 13 | 17 | | 99.999999 | | 5 min |
| 14 | 54 | | 99.999999 | | 5 min |

### Example 4: Preparation of Liquid Disinfectant Composition Using Silver Chloride, CTAC, and PVP-K30

100 mL of distilled water and cetrimonium chloride (CTAC) were added into a flask. The mixture was stirred until a clear solution was obtained using a magnetic stirrer. Then, silver chloride was added into the flask. The mixture was stirred for 15 minutes until a colorless or a slight hazy solution was obtained. PVP K-30 was added, and the mixture was stirred for an additional 30 minutes at room temperature until a colorless solution was obtained. Specific amounts of these reagents are shown below in Table 4.

TABLE 4
Light Stable Silver Compositions using Silver Chloride, CTAC, and PVP K-30

| Experiment | Formulation | Amount | | PVP K- | | Concentration |
|---|---|---|---|---|---|---|
| # | # | CTAC (g) | AgCl (g) | 30 | Water | of Silver |
| 1 | 38 | 5.714 | 0.02857 | 0.143 | 100 | 280 |
| 2 | 39 | 2.0 | 0.01 | 0.143 | 100 | 100 |
| 3 | 40 | 2.0 | 0.01 | 0.143 | 100 | 100 |
| 5 | 41 | 5.714 | 0.01 | 0.143 | 100 | 100 |
| | 42 | 2.0 | 0.0 | 0.143 | 100 | X |
| | 43 | 0.0 | 0.01 | 0.143 | 100 | 100 |

PVP K-30: Polyvinyl pyrrolidine K-30;
CTAC: cetrimonium chloride

### Example 5: Experimental Results for ASTM E-2315 Evaluation for Silver Chloride, Cetrimonium Chloride (CTAC), and PVP K-30

ASTM E-2315 evaluation was conducted as described above in Example 4 using the light stable disinfectant compositions as shown in Table 4. Table 5 shows the results of the ASTM-2315 tests.

TABLE 5
ASTM E-2315 Results

| Experiment | Formulation | S. aureus | E. coli | Pseudomonas | Exposure |
|---|---|---|---|---|---|
| # | # | Reduction | Reduction ( | aeruginosa | Time |
| 1 | 38 | 99.99999 | 99.999999 | | 5 min |
| 2 | 39 | | 99.999999 | | 5 min |
| 3 | 41 | | 99.9999 | | 5 min |
| | 42 | | 99.0 | | 5 min |
| | 43 | | 99.0 | | 5 min |

### Example 6: Preparation of Disinfectant Composition Using Silver Chloride, Sodium Lauryl Sulfate (SLS), and PVP K-30

100 mL of distilled water and sodium lauryl sulfate (SLS) were added into a flask. The mixture was stirred until a clear solution was obtained using a magnetic stirrer. Then, silver chloride was added into the flask. The mixture was stirred for 15 minutes until a colorless solution was obtained. PVP K-30 was added, and the mixture was stirred for an additional 30 minutes at room temperature until a colorless solution was obtained. Specific amounts of these reagents are shown below in Table 6.

TABLE 6
Light Stable Liquid Disinfectant Compositions using Silver
Chloride, SLS, and PVP K-30

| Experiment | Formulation | Amount | | | | Concentration |
|---|---|---|---|---|---|---|
| # | # | SLS (g) | AgCl (g) | PVP K-30 | Water | of Silver |
| 1 | 51 | 2.0 | 0.01 | 0.143 | 100 | 280 |
| 2 | 52 | 2.0 | 0.01 | 0.143 | 100 | 100 |
| 3 | 53 | 2.0 | 0.0 | 0.143 | 100 | X |

### Example 7: Experimental Results for ASTM E-2315 Evaluation for Silver Chloride, SLS, and PVP K-30

ASTM E-2315 evaluation was conducted as described above in Example 6 using the light stable disinfectant compositions as shown in Table 6. The results of these tests are shown in Table 7.

TABLE 7
ASTM E-2315 Results

| Experiment | Formulation | S. aureus | E. coli | Pseudomonas | Exposure |
|---|---|---|---|---|---|
| # | # | Reduction | Reduction ( | aeruginosa | Time |
| 1 | 51 | | 99.999999 | | 5 min |
| 2 | 52 | | 99.999999 | | 5 min |
| | 53 | | 99.999999 | | 5 min |

### Example 8: Preparation of Light Stable Liquid Disinfectant Comprising Trisodium Citrate, Silver Nitrate, and PVP K-30

The liquid disinfectant solution was prepared according to the following procedure. Trisodium citrate and distilled water were added into a round bottom flask with a magnetic stirring bar. The flask was flushed with nitrogen and wrapped with aluminum foil to protect the reaction from light. To this solution was added the water-soluble silver nitrate. After stirring for 5 minutes at room temperature, a colorless solution appeared. Then, PVP K-30 was added portion-wise. This mixture was stirred for an additional 10 minutes at room temperature. The pH of the solution was measured using a pH meter resulting in a pH of approximately 6.5-7.0. Yield: 99%. The light stable liquid disinfectant composition was stored in a plastic bottle and stored at ambient temperatures. Specific amounts of these reagents are shown below in Table 9.

TABLE 9
Light Stable Liquid Disinfectant Compositions using Silver Nitrate,
Trisodium Citrate, and PVP K-30

| | | Amount | | | | | |
|---|---|---|---|---|---|---|---|
| | | Trisodium | Amount | Amount | | Concentration | Nitrogen/ |
| Experiment | Formulation | Citrate | AgNO₃ | PVP | Water | of Silver | Dark |
| # | # | (g) | (g) | K-30 | (mL) | (ppm) | Use |
| 1 | 33 | 5.0 | 0.05 | 1.0 | 100 | 500 | yes |
| 2 | 34 | 2.0 | 0.05 | 1.0 | 100 | 500 | yes |
| 3 | 35 | 2.5 | 0.05 | 1.0 | 100 | 500 | yes |
| 4 | 36 | 2.0 | 0.1 | 2.0 | 100 | 1000 | yes |
| 5 | 37 | 9.0 | 0.1 | 2.0 | 100 | 1000 | yes |

ASTM E-2315 results using the above formulations 33-37 showed that when Escherichia coli was exposure for just 5 minutes, 99.999999% of the E. coli was eliminated thereby demonstrating instant killing activity of these formulation as compared to the control.

### Example 9: Preparation of Light Stable Disinfectants Comprising Water Soluble Metal Salts and PVP K-30

The liquid disinfectant solution was prepared according to the following procedure. Distilled water was added into a round bottom flask equipped with a magnetic stirring bar. To the stirred water was added the water-soluble metal salt. After stirring for 5 minutes at room temperature, until a colorless appeared. Then, the hydrophilic polymer was added portion wise. This mixture was stirred for an additional 10 minutes at room temperature. The pH of the solution was measured using a pH meter resulting in a pH of approximately 6.5-7.0. Yield: 99%. The light stable liquid disinfectant composition was stored in a plastic bottle and stored at ambient temperatures. Specific amounts of these reagents are shown below in Table 10.

This general example demonstrates that the order of addition is important to prepare the liquid disinfectant composition as a colorless solution.

TABLE 10
Light Stable Compositions using Water Soluble Metal Salts and PVP K-30

| | | Water | | | | |
|---|---|---|---|---|---|---|
| Experiment | Formulation | Soluble | Amount | PVP K- | Water | Concentration |
| # | # | Metal Salt | of Salt (g) | 30 (g) | (mL) | of Metal |
| 1 | 37 | Cu(OAc)₂ | 0.05 | 2.5 | 500 | 100 |
| 2 | 38 | AgOAc | 0.05 | 2.5 | 500 | 100 |
| 3 | 39 | AgOAc | 0.05 | 1.25 | 250 | 200 |
| 4 | 40 | Cu(OAc)₂ | 0.02 | 1.0 | 100 | 200 |
| 5 | 41 | Zn(OAc)₂ | 0.03 | 1.5 | 100 | 300 |
| 6 | 42 | Zn(OAc)₂ | 0.05 | 2.5 | 500 | 100 |
| 7 | 43 | Ag(lactate) | 0.025 | 1.25 | 250 | 100 |

### Example 9: Experimental Results for ASTM E-2315 Evaluation for Water Soluble Metal Salts and PVP K-30

ASTM E-2315 evaluation was conducted as described above in Example 3 using the light stable disinfectant compositions as shown in Table 10.

TABLE 10
ASTM E-2315 Results

| | | | | Pseudomonas | |
|---|---|---|---|---|---|
| | | S. aureus | E. coli | aeruginosa | |
| | | Reduction | Reduction | Reduction | |
| Experiment | Formulation | (exposure | (exposure | (exposure | Exposure |
| # | # | time 5 min) | time 5 min) | time 5 min) | Time |
| 1 | 37 | | 99.9 | | 5 min |
| 2 | 38 | | 99.9 | | 5 min |
| 3 | 42 | | 99 | | 5 min |
| 4 | 43 | | 99.999999 | | 5 min |
| 5 | 39 | | 99.999999 | | 5 min |

All the formulation killed more than 99% of the E. coli present.

### Example 10: Preparation of Light Stable Disinfectants Comprising Two or More Water Soluble Salts and PVP K-30

The liquid metal ion was prepared according to the following procedure. Distilled water was added into a round bottom flask equipped with a magnetic stirring bar. Two water-soluble metal salts were added to the stirred water. Stirring continued for 10 minutes at room temperature until a colorless appeared. Then, the hydrophilic polymer was added portion wise. This mixture was stirred for an additional 10 minutes at room temperature. The pH of the solution was measured using a pH meter resulting in a pH of approximately 6.5-7.0. Yield: 99%. The light stable liquid disinfectant composition was stored in a plastic bottle and stored at ambient temperatures. Specific amounts of these reagents are shown below in Table 11.

This general example demonstrates that the order of addition is important to prepare the liquid disinfectant composition as a colorless solution.

TABLE 11
Light Stable Disinfectants Comprising Two or More Water Soluble Salts and PVP K-30.

| | | | | | Amount of | | Amount of | | |
|---|---|---|---|---|---|---|---|---|---|
| | | First | | | Second | | Third | | |
| | | Water | Amount of | Second | Water | Third | Water | | |
| | | Soluble | First | Water | Soluble | Water | soluble | PVP | |
| Experiment | Formulation | Metal | Salt | Soluble | Salt | Soluble | salt | K-30 | Water |
| # | # | Salt | (g) | Salt | (g) | Salt | (g) | (g) | (mL) |
| 1 | 44 | Cu(OAc)₂ | 0.05 | Zn(OAc)₂ | 0.05 | X | X | 1.25 | 500 |
| 2 | 45 | Cu(OAc)₂ | 0.05 | AgOAc | 0.05 | X | X | 1.25 | 500 |
| 3 | 46 | AgOAc | 0.05 | Zn(OAc)₂ | 0.05 | X | X | 1.25 | 250 |
| 4 | 47 | Cu(OAc)₂ | 0.05 | Zn(OAc)₂ | 0.05 | AgOAc | 0.05 | 1.25 | 500 |

### Example 11: Experimental Results for ASTM E-2315 Evaluation for Two or More Water Soluble Metal Salts and PVP K-30

ASTM E-2315 evaluation was conducted as described above in Example 3 using the light stable disinfectant compositions as shown in Table 11.

TABLE 12
ASTM E-2315 Results

| | | | | Pseudomonas | |
|---|---|---|---|---|---|
| | | S. aureus | E. coli | aeruginosa | |
| | | Reduction | Reduction | Reduction | |
| Experiment | Formulation | (exposure | (exposure | (exposure | Exposure |
| # | # | time 5 min) | time 5 min) | time 5 min) | Time |
| 1 | 44 | | 99 | | 5 min |
| 1 | 47 | | 99.99 | | 5 min |
| 2 | 45 | | 99.999999 | | 5 min |

ASTM E-2315 results showed that when Escherichia coli was exposure for just 5 minutes, greater than 99% of the E. coli was eliminated thereby demonstrating instant killing activity of the liquid soap formulation as compared to the control.

### Example 12: Preparation of Hand Soap Formulation

The hand soap formulation was prepared as follows: In one flask, the liquid disinfectant composition was prepared, as described above, by contacting 2.50 g EDTA, 0.02 g of silver nitrate, and 1.0 g in PVP K-30 in 100 mL of distilled water forming a 200 ppm solution of silver.

90 mL distilled water and 1 g sodium lauryl sulfate were added into a separate flask using magnetic stirring. Into this solution was added the 200 ppm solution of silver, noted above, and 1 g of sodium chloride at room temperature. After stirring for 10 minutes at room temperature, 3.0 mL of cocamide monoethanolamide (ginnamide) was added. The mixture was stirred for an additional 10 minutes. The hand soap formulation was stored in a plastic bottle and stored at ambient temperatures. ASTM E-2315 results showed that when Escherichia coli was exposure for just 5 minutes, 99.9999% of the E. coli was eliminated thereby demonstrating instant killing activity of the liquid soap formulation as compared to the control.

### Example 13: Preparation of Disinfectant Composition Containing a Protein Material

95% ethanol was added into a round bottom flask equipped with a magnetic stirring bar. Once the stirring was initiated, silver nitrate was added. This mixture was stirred for 1 hour at ambient temperature. Then, zein powder was added in portions and the mixture was stirred for 1 hour resulting in a hazy mixture. The mixture was stored in a plastic bottle and stored at ambient temperature. Specific amounts of these reagents are shown below in Table 13.

TABLE 13
Disinfectant Compositions containing a Protein Material

| | | Zein | | 95% | |
|---|---|---|---|---|---|
| Experiment | Formulation | Powder | AgNO₃ | EtOH | ppm |
| # | # | (g) | (g) | (mL) | silver |
| 1 | 46 | 6 | X | 100 | X |
| 2 | 47 | 6 | 0.01 | 100 | 100 |
| 3 | 48 | 6 | 0.05 | 100 | 50 |
| 4 | 49 | 6 | 0.025 | 100 | 25 |
| 5 | 50 | 6 | 0.015 | 100 | 15 |
| 6 | 51 | 6 | 0.005 | 100 | 5 |

These mixtures of the liquid disinfectant composition were sprayed on fruit samples such as oranges, apples, and grapes. After these fruits were exposed to the disinfectant composition, the fruit retained 10 to 15% more moisture than the control samples not sprayed with the disinfectant composition for more than a week. ASTM E-2315 evaluation of the fruits versus Aspergillus brasiliensis may show that there would be a greater than 99% kill of the fungus.

### Example 14: Milk Preservative Experiment

To determine the effectiveness of the disinfectant composition as a preservative in whole milk, the disinfectant composition was added to whole milk at room temperature to determine the time until the milk spoiled. The disinfectant composition contained: 1.25 g EDTA, 0.01 g silver nitrate, and 0.5 g PVP K-30 in 100 mL of distilled water. Table 14 below shows the results from these experiments.

TABLE 14
Milk Preservative Experiment

| | | | Volume | | |
|---|---|---|---|---|---|
| | | | of 100 ppm | | |
| | | Volume | Disinfectant | | |
| Experiment | Vial | of Milk | Composition | Temperature | |
| # | # | (mL) | (mL) | (° C.) | Notes |
| 1 | 1 | 250 | 0 | 25 | Milk |
| | | | | | Spoiled |
| | | | | | within |
| | | | | | 24 h |
| 1 | 2 | 250 | 0.5 | 25 | Milk |
| | | | | | unspoiled |
| | | | | | after |
| | | | | | 24 h |
| 2 | 1 | 3.0 | 0 | 25 | Spoiled |
| | | | | | 7 h |
| 2 | 2 | 3.0 | 0.1 | 25 | Spoiled |
| | | | | | 17 h |
| 2 | 3 | 3.0 | 0.2 | 25 | Spoiled |
| | | | | | 20 h |
| 2 | 4 | 3.0 | 0.3-0.4 | 25 | Spoiled |
| | | | | | 22-23 h |
| 2 | 5 | 3.0 | 0.5 | 25 | Spoiled |
| | | | | | 48-50 h |
| 2 | 6 | 3.0 | 0.6 | 25 | Spoiled |
| | | | | | 57 h |
| 3 | 1 | 3.0 | 0 | 25 | Spoiled |
| | | | | | in |
| | | | | | 7 h |
| 3 | 2 | 3.0 | 0.1 | 25 | Spoiled |
| | | | | | in |
| | | | | | 17- |
| 3 | 3 | 3.0 | 0.2 | 25 | Spoiled |
| | | | | | in |
| | | | | | 20 h |
| 3 | 4 | 3.0 | 0.3 | 25 | Spoiled |
| | | | | | 23-25 h |
| 3 | 5 | 3.0 | 0.4 | 25 | Spoiled |
| | | | | | 58-67 h |
| 3 | 6 | 3.0 | 0.5 | 25 | Not |
| | | | | | Spoiled |
| | | | | | after |
| | | | | | 72 h |
| 3 | 7 | 3.0 | 0.6 | 25 | Not |
| | | | | | Spoiled |
| | | | | | after |
| | | | | | 72 h |
| 3 | 8 | 3.0 | 0.7 | 25 | Not |
| | | | | | Spoiled |
| | | | | | after |
| | | | | | 72 h |

As can be seen in the above table, the addition of amounts of the disinfectant composition extended the duration of the milk until the milk spoiled.

### Example 14: Egg Preservation Experiment

To determine the effectiveness of the disinfectant composition as a preservative on eggs, the disinfectant composition was sprayed on poultry fresh eggs at room temperature to determine the time until the eggs spoiled. When the eggs floated in water, the eggs lost their freshness. Two disinfectant composition were evaluated: Liquid Disinfectant Composition A containing: 1.25 g EDTA, 0.01 g silver nitrate, and 0.5 g PVP K-30 in 100 mL of distilled water; Liquid disinfectant Composition B containing: 0.01 g silver chloride, 0.1 g CTAC, 0.1 g PVP K-30, and 100 mL of distilled water. Table 14 below shows the results from these experiments.

TABLE 14
Egg Preservative Experiment

| | | Sprayed with | | |
|---|---|---|---|---|
| Experiment | | Disinfectant | Duration | |
| # | Composition | Composition | (days) | Observation |
| 1 | A | no | 19 | Untreated |
| | | | | eggs float |
| | | | | in water |
| 1 | A | yes | 26 | Treated |
| | | | | eggs float |
| | | | | in water |
| 2 | A | no | 12 | Untreated |
| | | | | eggs float |
| | | | | in water |
| 2 | A | yes | 14 | Treated |
| | | | | eggs float |
| | | | | in water |
| 3 | A | no | 9 | Untreated |
| | | | | eggs float |
| | | | | in water |
| 3 | A | yes | 12 | Treated |
| | | | | eggs float |
| | | | | in water |
| 4 | B | no | 15 | Untreated |
| | | | | eggs float |
| 4 | B | yes | 15 | Treated |
| | | | | eggs sink |
| | | | | in water |

As can be seen in the above table, eggs sprayed with the disinfectant composition retained their freshness for more than 2 days as compared to eggs not treated with the disinfectant composition,

### Example 16: Evaluation of the Liquid Disinfectant Composition in the Fosun Covid-19 RT-PCR Test Against Viruses

This example shows the liquid disinfectant composition in the Fonsum Pharma test against a Covid-19 RT-PCR Detection Kit. The disinfectant composition contained: 1.25 g EDTA, 0.01 g silver nitrate, and 0.5 g PVP K-30 in 100 mL of distilled water.

5%, 10%, 25%, 50%, 75%, and 100% concentrations were used in the evaluation. The liquid disinfectant composition has the ability to inhibit the growth of SARS-CoV-2 in vitro. MRIGlobal utilized the USA-WA1/2020 strain of the virus, acquired from BEI Resources (NR-52281). This was propagated in Vero E6 cells (ATCC CRL-1586); these cells were also used for the neutralization assay. Vero E6 cells were cultured in growth media consisting of Dulbeco's Modified Eagle Medium/F12 supplemented with 5% FBS (Fetal Bovine Serum), and PSN (penicillin, streptomycin, and neomycin).

The Vero E6 cells were plated on 96-well plates 1-3 days before the assay and were allowed to grow to ˜60%-70% confluence. On the day of the assay, 150 μl of stock virus was added to 1.35 ml of liquid disinfectant composition that was shaken before adding to tubes. Immediately after virus addition or 10 minutes after addition, 1.5 ml of 0.5% sodium thiosulfate was added in an attempt to neutralize any cytotoxic chemicals in the solution. Samples were added to an empty 96-well plate and diluted 1:10 down the plate in DMEM/F12. Samples were taken after liquid disinfectant composition had settled and after agitation in case there was any difference in viral recovery. Upon plate examination, agitated samples showed no increase in CPE but an increase in cytotoxicity so were not assessed further. These dilutions were transferred to a plate of Vero cells with media removed. After at least 15 minutes, DMEM/F12 supplemented with FBS was added to cells to feed them for the next 3 days. This incubation period of at least 15 minutes is to allow the virus to adsorb to cells without interference from FBS. Cytotoxicity controls of the test articles without virus added were also performed. The assay was executed in three technical replicates and five pipetting replicates for each condition.

After 5 days, cells were examined for the presence of cytopathic effect (CPE) associated with viral presence and replication. Examination is done using a microscope (10× objective to view the entire well at once) and observing the morphology of the cells. Healthy Vero cells have a semitransparent appearance with pinched or rounded ends in a monolayer of cells with little to no space between cells. Dead cells displaying CPE are often not adhered to the plate, round and much smaller than living cells. Considerable empty space can be seen on the bottom of the plate from where cells have detached. Any well displaying CPE is marked as positive, whether the whole well, or only a portion is affected, because this is indicative of viable virus.

Cytotoxicity was observed at the first two dilutions of all three treatments but not from controls. CPE was observed past cytotoxicity for the immediate contact test so it can be inferred that virus was present in the cytotoxic wells also. For the 10-minute contact time, no CPE was observed in wells past those showing cytotoxicity, so it is possible that there was no virus in the wells with cytotoxicity. However, since the cells died, there is no way to determine viral presence in this assay. All uninfected controls remained healthy and did not display any CPE throughout the 5-day observation period. Results were calculated using the Reed & Muench Calculator (produced by BD Lindenbach from “Measuring HCV infectivity produced in cell culture and in vivo” Methods Mol Biol. (2009) 510:329-36). Results are shown as Log reduction relative to timed controls as well as a percent reduction of SARS-CoV-2 infectivity.

Based on these experiments, it is concluded that SARS-CoV-2 infection of Vero cells is inhibited by liquid disinfectant composition upon immediate contact and even further inhibited after 10 minutes of contact by 99% or greater. It is important to note that the cytotoxicity observed limits the enumeration of virus and, therefore, it cannot be said whether virus was present in those wells which showed cytotoxicity. Thus, actual viral reduction is potentially greater than reported for 10 minutes which is than the 99% reduction of viruses was found from 5% concentrations and above.

### Example 17: Evaluation of the Liquid Disinfectant Composition in the ASTM E 2315 Test Against Fungi

This example shows the liquid disinfectant composition in the ASTM E-2315 test against a series of fungi. The tests would be conducted in a similar fashion as disclosed above except using the fungi: Trichophyton rubrum. “The disinfectant composition contained: 1.25 g EDTA, 0.01 g silver nitrate, and 0.5 g PVP K-30 in 100 mL of distilled water. The evaluation of these results is expected to show that more than a 99.999999% reduction of fungi was found.

### Example 18: Preparation of Other Liquid Disinfectant Compositions for Fabrics

A liquid disinfectant composition is prepared similarly according to Example 1, noted above. The additives consisting of silica powder and titania powder were added after the liquid disinfectant composition was prepared. The final liquid metal disinfectant contains the following components which is used to disinfect fabrics:

Silver nitrate, 2 wt %

Poly(vinyl pyrrolidone) K30, 4 wt %

Ethylenediaminetetraacetic acid (EDTA), 1 wt %

Silica powder, 0.5 wt %

Titania powder, 1 wt %

Water, remainder

### Example 19: Preparation of Other Liquid Disinfectant Compositions

A general disinfectant composition is prepared by mixing the starting components together, in a manner substantially as described in Example 8. The additives Triton X-100 and melamine were added after the liquid disinfectant composition was prepared. The final disinfectant composition is as follows:

Silver nitrate, 2 wt %

Poly(vinyl pyrrolidone) K-30, 4 wt %

Trisodium citrate, 10 wt %

Triton X-100 (polyoxyethylene octyl phenyl ether), 0.1 wt %

Melamine, 1 wt %

Water, remainder

### Example 20: Stability Studies of Liquid Disinfectant Composition

The following stability study was conducted to identify the liquid disinfectant compositions would maintain their color for an extended period of time at room temperature and under normal light conditions under normal atmospheric conditions in a sealed container.

The liquid compositions contain 100 mL distilled water, 1.25 g EDTA, 0.01 g silver nitrate, and 0.5 g PVP K-30 (100 ppm) or 100 mL distilled water, 2.0 g EDTA, 1.0 g silver nitrate, and 4.0 g PVP K-30 (1000 ppm). The change in color from a colorless solution to a colored solution or black flakes in solution would indicate the original amount of silver present in the compositions would be diminished and the formation of other silver salts (such as silver oxide and silver carbonate) were present. The data for these stability studies is shown in Table 15 below:

TABLE 15
Stability Studies

| | | Color or | | |
|---|---|---|---|---|
| Experiment | Ppm | Appearance | Number | |
| # | Concentration | Change | of Days | Temperature |
| 1 | 100 | Colorless | Initially | Room |
| | | Solution | | temperature |
| 2 | 100 | Colorless | 7 Days | Room |
| | | Solution | | temperature |
| 3 | 100 | Colorless | 100 Days | Room |
| | | Solution | | temperature |
| 4 | 1000 | Colorless | Initially | Room |
| | | Solution | | Temperature |
| 5 | 1000 | Some black | 7 days | Room |
| | | flakes in a | | Temperature |
| | | colorless | | |
| | | solution | | |

The data in the above tale shows that the liquid disinfectant composition at a 100 ppm level is heat and light stable.

### Example 21: Liquid Disinfectant Composition on Fresh Cut Flowers

Into a round bottom flask was added 100 mL of distilled water and a magnetic stirring bar. Once the stirring was initiated, 0.01 g of silver nitrate may be added. This mixture may be stirred for 5 minutes at ambient temperature until a homogeneous solution would be obtained. Then, 0.5 g of glycerol and 0.5 g of coconut oil may be added in portions and the mixture may be stirred for 1 hour resulting in a hazy mixture. The mixture may be stored in a plastic bottle and stored at ambient temperature.

Fresh cut flowers may be coated with the liquid disinfectant composition detailed above. The fresh cut treated flowers would be compared to fresh cut flowers just soaked in water. The expected results would show that the treated flowers would not wilt as compared to the untreated flowers for more than 10 days.

### Example 22: Durability Studies of Liquid Disinfectant Composition

The durability test was designed to identify the number of times a surface can be wiped under pressure and prevent growth of pathogens on the surface after the light stable disinfectant was applied.

The liquid composition contains 100 mL distilled water, 1.25 g EDTA, 0.01 g silver nitrate, and 0.5 g PVP K-30 having a 100 ppm concentration of silver. Five test solutions were evaluated: 10 mL liquid disinfectant composition, sodium hypochlorite, SaniDate (peracetic acid), and Lysol.

This 100 ppm solution of silver solution was loaded into a sprayer and sprayed onto 1×1″ sterilized aluminum or stainless-steel coupons. The aluminum coupons were wiped with a sterilized non-scratch scouring sponge or a soft cloth with a 1 kg weight on top of the non-scratch scouring sponge or a soft cloth then autoclaved. After the aluminum coupons had cooled to room temperature, the test solutions were sprayed onto the coupons. After the aluminum coupon was wiped 160 times, a sample of the surface was taken from the aluminum coupon and tested for E. coli using the ASTM E-2315 test. The results, shown below in Table 16, show that the level of pathogens did not increase while commercial disinfectant composition showed less durability as compared to the liquid disinfectant composition.

TABLE 16
Durability Evaluation

| Experiment # | Coupons | Solution | Times | results |
|---|---|---|---|---|
| 1 | Aluminum | AgNO₃, | 160 times | 99.999999% |
| | | EDTA, | | reduction of |
| | | PVP K-30 | | E. coli. |
| 2 | Aluminum | Lysol | 80 Times | 99.9999% |
| | | | | reduction of |
| | | | | E. coli. |
| 3 | Aluminum | Santidate | 40 times | 99.999% |
| | | | | reduction |
| 4 | Aluminum | Sodium | 40 times | 0% |
| | | hypochlorite | | reduction |

This test demonstrates that the liquid disinfectant composition prevents pathogens from growing on a surface wiped more than 160 times.

What is claimed is:

1. A liquid disinfectant composition comprising:
a) 0.001 wt % to 5.0 weight % (wt %) of at least one metal ion;
b) 0.1 wt % to 5.0 wt % of at least one hydrophilic polymer; and
c) 89.9 wt % to 99.9 wt % of at least one solvent;
wherein the liquid disinfectant does not comprise nanoparticles.

2. The liquid disinfectant composition of claim 1, wherein the composition further comprises at least one chelating agent, at least one surfactant, or a combination of at least one chelating agent and at least one surfactant.

3. The liquid disinfectant composition of claim 1, wherein the at least one chelating agent, the at least one surfactant, or a combination of the at least one chelating agent and the at least one surfactant, when present, has a weight % of 1.0 wt % to 20.0 wt %.

4. The liquid disinfectant composition of claim 1, wherein the composition may further comprise at least one additive.

5. The liquid disinfectant composition of claim 4, wherein the additive, when present, ranges from about 0.1 wt % to about 10 wt % of the total weight of the liquid disinfectant composition.

6. The liquid disinfectant composition of claim 4, wherein the additive is selected from a group consisting of a wetting agent, a binding agent, an essential oil, an emulsifier, a protein material, and a combination thereof.

7. The liquid disinfectant composition of claim 1, wherein the composition can maintain efficacy and/or pathogenic sterility for up to 60 days on a variety of surfaces.

8. The liquid disinfectant composition of claim 1, wherein the at least one metal ion is from a water-soluble metal salt.

9. The liquid disinfectant composition of claim 8, wherein the at least one metal salt is selected from a group consisting of a silver salt, a copper salt, a zinc salt, a gold salt, a cobalt salt, a nickel salt, a zirconium salt, a molybdenum salt, a palladium salt, and combinations thereof.

10. The liquid disinfectant composition of claim 9, wherein the at least one metal salt is selected from a group consisting of a silver salt, a copper salt, a zinc salt, and a combination thereof.

11. The liquid disinfectant composition of 9, wherein the at least one metal salt is a combination of a silver salt and a copper salt.

12. The liquid disinfectant composition of claim 9, wherein the at least one metal salt is a combination of a silver salt and a zinc salt.

13. The liquid disinfectant composition of claim 9, wherein the at least one metal salt is a combination of a copper salt and a zinc salt.

14. The liquid disinfectant composition of claim 9, wherein the at least one metal salt is a combination of a copper salt, a zinc salt, and a silver salt.

15. The liquid disinfectant composition of claim 1, wherein the hydrophilic polymer is selected from a group consisting of a polyacrylamide, a poly(acrylamide-co-acrylic acid), poly(vinyl alcohol), poly(vinyl pyrrolidone), poly (ethylene oxide), water soluble polyurethane, carboxy methyl cellulose, lipids, glycerolipids, fatty acid lipid polymers, oligosaccharides, glycerols, and combinations thereof.

16. The liquid disinfectant composition of claim 15, wherein the hydrophilic polymer is poly(vinyl pyrrolidone) (PVP) and has a high or low molecular weight.

17. The liquid disinfectant composition of claim 16, wherein the hydrophilic polymer is poly(vinyl pyrrolidone) K-30 (PVP K-30) or poly(vinyl pyrrolidone) K-90 (PVP K-90).

18. The liquid disinfectant composition of claim 17, wherein the chelating agent is selected from a group consisting of citric acid, a citrate salt, ascorbic acid, an ascorbate salt, a polyaminocarboxylic acid, a salt of a polyaminocarboxylic acid, an organic compound, a salt of an organic compound, and combinations thereof.

19. The liquid disinfectant composition of claim 18, wherein the polyaminocarboxylic acid is selected from a group consisting of iminodiacetic acid (IDA), nitrilotriacetic acid (NTA), ethylenediaminetetraacetic acid (EDTA), diethylenetriaminepentaacetic acid (DTPA), ethylene glycol-bis(β-aminoethyl ether)-N,N,N′,N′-tetraacetic acid (EGTA), 1,2-bis(o-aminophenoxy)ethane-N,N,N′,N′-tetraacetic acid (BAPTA), 1,4,7-triazacyclononane-1,4,7-triacetic acid (NOTA), 2,2′,2″,2′″-(1,4,7,10-tetraazacyclododecane-1,4,7,10-tetrayl)tetraacetic acid (DOTA), (2S)-1-[(3S)-3-{[(3S)-3-amino-3-carboxypropyl]amino}-3-carboxypropyl]azetidine-2-carboxylic acid, ethylenediamine-N,N′-bis(2-hydroxyphenylacetic acid (EDDHA), ethylenediamine-N,N′-disuccinic acid (EDDS), and combinations thereof.

20. The liquid disinfectant composition of claim 19, wherein the polyaminocarboxylic acid is ethylenediaminetetraacetic acid, a disodium salt of ethylenediaminetetraacetic acid, or a combination thereof.

21. The liquid disinfectant composition of claim 20, wherein the polyaminocarboxylic acid is ethylenediaminetetraacetic acid.

22. The liquid disinfectant composition of claim 18, wherein the chelating agent is sodium citrate.

23. The liquid disinfectant composition of claim 1, wherein the surfactant is a cationic surfactant, an anionic surfactant, a nonionic surfactant, a zwitterionic surfactant, or a combination thereof.

24. The liquid disinfectant composition of claim 23, wherein the surfactant is selected from a group consisting of benzalkonium chloride, cetalkonium chloride, cetrimonium bromide, and cetrimonium chloride, sodium lauryl sulfate, and combinations thereof.

25. The liquid disinfectant composition of claim 24, wherein the surfactant is cetrimonium chloride.

26. The liquid disinfectant composition of claim 24, wherein the surfactant is sodium lauryl sulfate.

27. The liquid disinfectant composition of claim 1, wherein the at least one metal salt has a mole % ranging from about 0.001 mole % to about 0.05 mole %.

28. The liquid disinfectant composition of claim 1, wherein the at least one hydrophilic polymer has a mole % ranging from 0.0000001 mole % to about 0.001 mole %.

29. The liquid disinfectant composition of claim 1, wherein the at least one solvent has a mole % of from about 97.5 mole % to about 99.9 mole %.

30. The liquid disinfectant composition of claim 1, wherein the least one chelating agent, the at least one surfactant, or a combination of the at least one chelating agent and the at least one surfactant, when present, has a mole ratio of 0.1 mole % to about 1.0 mole %.

31. The liquid disinfectant composition of claim 1, wherein the least one chelating agent, the at least one surfactant, or a combination of the at least one chelating agent and the at least one surfactant to the at least one metal has a weight ratio ranging from about 100.0:1.0 to about 150.0:1.0.

32. The liquid disinfectant composition of claim 31, wherein the weight ratio of the least one chelating agent, the at least one surfactant, or a combination of the at least one chelating agent and the at least one surfactant to the at least one metal salt is about 125.0:1.0.

33. The liquid disinfectant composition of claim 1, wherein the hydrophilic polymer to the one or more metal salt has a weight ratio ranging from about 30.0:1.0 to about 70.0:1.0.

34. The liquid disinfectant composition of claim 33, wherein the weight ratio of the hydrophilic polymer to the at least one metal salt containing composition is about 50.0:1.0.

35. The liquid disinfectant composition of claim 1, wherein the weight to volume ratio of the least one chelating agent, the at least one surfactant, or a combination of the at least one chelating agent and the at least one surfactant to the at least one solvent ranges from about 1.0:100.0 to about 10.0:100.0.

36. The liquid disinfectant composition of claim 1, wherein the weight to volume ratio of the at least one metal salt to the at least one solvent ranges from about 0.001:100.0 to about 0.1:100.0.

37. The liquid disinfectant composition of claim 1, wherein the weight to volume ratio of the hydrophilic polymer to the solvent ranges from about 0.1:100.0 to about 5.0:100.0.

38. The liquid disinfectant composition of claim 1, wherein the disinfectant composition has a pH of about 6 to about 8 in water.

39. The liquid disinfectant composition of claim 1, wherein the composition is non-toxic and non-corrosive.

40. The liquid disinfectant composition of claim 1, wherein the composition is light and heat stable.

41. The liquid disinfectant composition of claim 1, wherein the at least one hydrophilic polymer prevents oxidation and/or moisture of the at least one metal ion.

42. The liquid disinfectant composition of claim 1, wherein the maintains contact on a variety of surfaces.

43. The composition of claim 1, wherein the liquid disinfectant composition exhibits antimicrobial properties, antibacterial properties, antifungal properties, antiviral properties, or a combination thereof against a variety of pathogens.

44. The liquid disinfectant composition of claim 1, wherein the composition achieves a greater than a 99% kill rate on a variety of surfaces in less than 5 minutes.

45. The liquid disinfectant composition of claim 1, wherein the composition maintains the efficacy for up to 60 days.

46. The liquid disinfectant composition of claim 1 comprising:
a) 0.01 wt % to 1.0 weight % (wt %) of at least one metal ion;
b) 0.1 wt % to 5.0 wt % of PVP K-30 or PVP K-90; and
c) 90 wt % to 99.9 wt % water;
wherein the liquid disinfectant composition does not comprise nanoparticles.

47. The disinfectant composition of claim 46, wherein the at least one metal salt is selected from the group consisting of a silver salt; a copper salt; a silver salt and a copper salt; a silver salt and a zinc salt; a copper salt and a zinc salt; and a silver salt, a copper salt, and a zinc salt.

48. The disinfectant composition of claim 46, wherein the hydrophilic polymer is PVP K-30 or PVP K-90.

49. The disinfectant composition of claim 46, wherein the composition further comprises at least one chelating agent, at least one surfactant, or a combination of at least one chelating agent and at least one surfactant in 1.0 wt % to 10.0 wt %.

50. The disinfectant composition of claim 46, wherein the at least one chelating agent, at least one surfactant, or a combination of at least one chelating agent and at least one surfactant is selected from a group consisting of at least one chelating agent, at least one surfactant, or a combination of at least one chelating agent and at least one surfactant, sodium citrate, sodium lauryl sulfate, cetrimonium chloride, and combinations thereof.

51. A method for preparing a light stable, non-corrosive, and non-toxic liquid disinfectant composition for killing pathogens comprising:
a) contacting at least one metal ion in at least solvent forming a mixture; and
b) contacting the mixture from step (a) with at least one hydrophilic polymer forming the liquid disinfectant composition;
wherein the liquid disinfectant does not comprise nanoparticles; and wherein the hydrophilic polymer prevents oxidation of the at least one metal ion and maintains contact with a variety of surfaces.

52. The method of claim 51, further comprising contact at least one chelating agent, at least one surfactant, or a combination thereof in step (a).

53. The method of claim 51, wherein the composition may further comprise at least one additive.

54. The method of claim 53, wherein the additive is selected from a group consisting of a wetting agent, a binding agent, an essential oil, an emulsifier, a protein material, and a combination thereof.

55. The method of claim 51, wherein the composition can maintain efficacy for at least 30 or more days on a variety of surfaces.

56. The method of claim 51, wherein the at least one metal ion is derived from a water-soluble metal salt.

57. The method of claim 56, wherein the at least one metal salt is selected from a group consisting of a silver salt, a copper salt, a zinc salt, a gold salt, a cobalt salt, a nickel salt, a zirconium salt, a molybdenum salt, a palladium salt, and combinations thereof.

58. The method of claim 56, wherein the at least one metal salt is selected from a group consisting of a silver salt, a copper salt, a zinc salt, and a combination thereof.

59. The method of 57, wherein the at least one metal salt is a combination of a silver salt and a copper salt.

60. The method of claim 57, wherein the at least one metal salt is a combination of a silver salt and a zinc salt.

61. The method of claim 57, wherein the at least one metal salt is a combination of a copper salt and a zinc salt.

62. The method of claim 57 wherein the at least one metal salt is a combination of a copper salt, a zinc salt, and a silver salt.

63. The method of claim 51, wherein the hydrophilic polymer is selected from a group consisting of a polyacrylamide, a poly(acrylamide-co-acrylic acid), poly(vinyl alcohol), poly(vinyl pyrrolidone), poly (ethylene oxide), water soluble polyurethane, carboxy methyl cellulose, lipids, glycerolipids, fatty acid lipid polymers, oligosaccharides, glycerols, and combinations thereof.

64. The method of claim 63, wherein the hydrophilic polymer is poly(vinyl pyrrolidone) (PVP) is a high or low molecular weight poly(vinyl pyrrolidone).

65. The method of claim 64, wherein the hydrophilic polymer is poly(vinyl pyrrolidone) K-30 (PVP K-30) or poly(vinyl pyrrolidone) K-90 (PVP K-90).

66. The method of claim 51, wherein the chelating agent is selected from a group consisting of citric acid, a citrate salt, ascorbic acid, an ascorbate salt, a polyaminocarboxylic acid, a salt of a polyaminocarboxylic acid, an organic compound, a salt of an organic compound, and combinations thereof.

67. The method of claim 66, wherein the polyaminocarboxylic acid is selected from a group consisting of iminodiacetic acid (IDA), nitrilotriacetic acid (NTA), ethylenediaminetetraacetic acid (EDTA), diethylenetriaminepentaacetic acid (DTPA), ethylene glycol-bis(β-aminoethyl ether)-N,N,N′,N′-tetraacetic acid (EGTA), 1,2-bis(o-aminophenoxy)ethane-N,N,N′,N′-tetraacetic acid (BAPTA), 1,4,7-triazacyclononane-1,4,7-triacetic acid (NOTA), 2,2′,2″,2′″-(1,4,7,10-tetraazacyclododecane-1,4,7,10-tetrayl)tetraacetic acid (DOTA), (2S)-1-[(3S)-3-{[(3S)-3-amino-3-carboxypropyl]amino}-3-carboxypropyl]azetidine-2-carboxylic acid, ethylenediamine-N,N′-bis(2-hydroxyphenylacetic acid (EDDHA), ethylenediamine-N,N′-disuccinic acid (EDDS), and combinations thereof.

68. The method of claim 67, wherein the polyaminocarboxylic acid is ethylenediaminetetraacetic acid, a disodium salt of ethylenediaminetetraacetic acid, or a combination thereof.

69. The method of claim 68, wherein the polyaminocarboxylic acid is ethylenediaminetetraacetic acid.

70. The method of claim 66, wherein the chelating agent is sodium citrate.

71. The method of claim 51, wherein the surfactant is a cationic surfactant, an anionic surfactant, a nonionic surfactant, a zwitterionic surfactant, or a combination thereof.

72. The method of claim 71, wherein the surfactant is selected from a group consisting of benzalkonium chloride, cetalkonium chloride, cetrimonium bromide, and cetrimonium chloride, sodium lauryl sulfate, and combinations thereof.

73. The method of claim 72, wherein the surfactant is cetrimonium chloride.

74. The method of claim 72, wherein the surfactant is sodium lauryl sulfate.

75. The method of claim 51, wherein the binding agent is selected from a group consisting of melamine, thiols, fatty acids, and combinations thereof.

76. The method of claim 51, wherein the at least one metal salt has a mole % ranging from about 0.001 mole % to about 0.05 mole %.

77. The method of claim 51, wherein the at least one hydrophilic polymer has a mole % ranging from 0.0000001 mole % to about 0.001 mole %.

78. The method of claim 51, wherein the at least one solvent has a mole % of from about 97.5 mole % to about 99.9 mole %.

79. The method of claim 51, wherein the least one chelating agent, the at least one surfactant, or a combination of the at least one chelating agent and the at least one surfactant to the at least one metal salt has a weight ratio ranging from about 100.0:1.0 to about 150.0:1.0.

80. The method of claim 79, wherein the weight ratio of the least one chelating agent, the at least one surfactant, or a combination of the at least one chelating agent and the at least one surfactant to the at least one metal salt is about 125.0:1.0.

81. The method of claim 51, wherein the hydrophilic polymer to the at least one metal salt has a weight ratio ranging from about 30.0:1.0 to about 70.0:1.0.

82. The method of claim 81, wherein the weight ratio of the hydrophilic polymer to the at least one metal salt is about 50.0:1.0.

83. The method of claim 51, wherein the weight to volume ratio of the least one chelating agent, the at least one surfactant, or a combination of the at least one chelating agent and the at least one surfactant to at least one solvent ranges from about 1.0:100 to about 10.0:100.

84. The method of claim 51, wherein the weight to volume ratio of the hydrophilic polymer to at least one solvent ranges from about 0.1:100.0 to about 5.0:100.0.

85. The method of claim 51, wherein the weight to volume ratio of the at least one metal salt to at least one solvent ranges from about 0.001:100.0 to about 0.1:100.0.

86. The method of claim 51, wherein the additive, when present, ranges from about 0.1 wt % to about 10.0 wt % of the total weight of the liquid disinfectant composition.

87. The method of claim 51, wherein the method is conducted at a temperature of about 0° C. to about 50° C.

88. The method of claim 87, wherein the method is conducted at room temperature (˜23° C.).

89. A method of disinfecting and/or maintaining the pathogenic sterility of an article comprising contacting the article with the liquid disinfecting composition of claim 1.

90. The method of claim 89, wherein the method kills greater than 99% of pathogens on the surface of the article.

91. The method of claim 89, wherein the cleaned and/or disinfected article maintains the reduction of pathogens and/or efficacy for greater than 1 hour day.

92. The method of claim 89, wherein the cleaned and/or disinfected article can be wiped at least 160 times with a non-scratch scouring sponge or a soft cloth under 1 kg of weight and prevent pathogens from growing on the article.

93. A method of cleaning a surface of an article comprising contacting the article with the liquid disinfecting composition of claim 1.
